SEQUENCE LISTING (44 sequences)

SEQ ID NO: 1 - DNA, 784 bp, Artificial Sequence
  expression cassette of the construct Nt-ptDP-ACT+SHR
gctcccccgccgtcgttcaatgagaatggataagaggctcgtgggattgacgtgagggggcagggatggctatatttctgggagcgaactccgggcgaatacgaagcgcttggatacgcatgcctgcaggcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgacatgcaatgtcatccatcatgtcgtgtacattgtccacgtccaagtttttatgggctttcttaagagcttcagctgcatttttcatagattccaatactgtggtgttcgtactagctccctccagagcttctcgttgaagttcaatagtagttaaagtgccatctatttgcaactgatttttttctaatcgcttcttccgcttcagcgcttgcatggccgctcagatccccgggtaccgagctcgtatccaagcgcttcgtattcgcccggagttcgctcccagaaatatagccatccctgccccctcacgtcaatcccacgagcctcttatccattctcattgaacgacggcgggggagc

SEQ ID NO: 2 - DNA, 817 bp, Artificial Sequence
  expression cassette of the construct Nt-ptSL-ACT+SHR
gctcccccgccgtcgttcaatgagaatggataagaggctcgtgggattgacgtgagggggcagggatggctatatttctgggagcgaactccgggcgaatacgaagcgcttggatacgcatgcgggtgggtggaaaaccacccacccctgcaggcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgacatgcaatgtcatccatcatgtcgtgtacattgtccacgtccaagtttttatgggctttcttaagagcttcagctgcatttttcatagattccaatactgtggtgttcgtactagctccctccagagcttctcgttgaagttcaatagtagttaaagtgccatctatttgcaactgatttttttctaatcgcttcttccgcttcagcgcttgcatggccgctcagatcccgcacgctcctaatggagcgtgcggtatccaagcgcttcgtattcgcccggagttcgctcccagaaatatagccatccctgccccctcacgtcaatcccacgagcctcttatccattctcattgaacgacggcgggggagc

SEQ ID NO: 3 - DNA, 1609 bp, Artificial Sequence
  expression cassette of the construct Nt-ptHP-ACT+SHR
gctcccccgccgtcgttcaatgagaatggataagaggctcgtgggattgacgtgagggggcagggatggctatatttctgggagcgaactccgggcgaatacgaagcgcttggatacgcatgcctgcaggcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgacatgcaatgtcatccatcatgtcgtgtacattgtccacgtccaagtttttatgggctttcttaagagcttcagctgcatttttcatagattccaatactgtggtgttcgtactagctccctccagagcttctcgttgaagttcaatagtagttaaagtgccatctatttgcaactgatttttttctaatcgcttcttccgcttcagcgcttgcatggccgctcagatctggtacggaccgtactactctattcgtttcaatatatttatttgtttcagctgactgcaagattcaaaaatttctttattattttaaattttgtgtcactcaaaaccagataaacaatttgatatagaggcactatatatatacatattctcgattatatatgtaaatgagttaaccttttttttccacttaaaattatatagggggatccccggggagcggccatgcaagcgctgaagcggaagaagcgattagaaaaaaatcagttgcaaatagatggcactttaactactattgaacttcaacgagaagctctggagggagctagtacgaacaccacagtattggaatctatgaaaaatgcagctgaagctcttaagaaagcccataaaaacttggacgtggacaatgtacacgacatgatggatgacattgcatgtcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgcgagctcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctac

SEQ ID NO: 4 - DNA, 549 bp, Artificial Sequence
  expression cassette of the construct St-ptDP-ACT
gctcccccgccgtcgttcaatgagaatggataagaggctcgtgggattgacgtgagggggcagggatggctatatttctgggagcgaactccgggcgaatacgaagcgcttggatacgcatgcctgcaggcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgacatgagctcgtatccaagcgcttcgtattcgcccggagttcgctcccagaaatatagccatccctgccccctcacgtcaatcccacgagcctcttatccattctcattgaacgacggcgggggagc

SEQ ID NO: 5 - DNA, 472 bp, Artificial Sequence
  expression cassette of the construct St-ptDP-SHR
gctcccccgccgtcgttcaatgagaatggataagaggctcgtgggattgacgtgagggggcagggatggctatatttctgggagcgaactccgggcgaatacgaagcgcttggatacgcatgcctgcaggcaatgtcatccatcatgtcgtgtacattgtccacgtccaagtttttatgggctttcttaagagcttcagctgcatttttcatagattccaatactgtggtgttcgtactagctccctccagagcttctcgttgaagttcaatagtagttaaagtgccatctatttgcaactgatttttttctaatcgcttcttccgcttcagcgcttgcatggccgctcgagctcgtatccaagcgcttcgtattcgcccggagttcgctcccagaaatatagccatccctgccccctcacgtcaatcccacgagcctcttatccattctcattgaacgacggcgggggagc

SEQ ID NO: 6 - DNA, 784 bp, Artificial Sequence; identical to SEQ ID NO: 1
  expression cassette of the construct St-ptDP-ACT+SHR

SEQ ID NO: 7 - DNA, 905 bp, Artificial Sequence
  expression cassette of the construct St-ptDP-EPI+PMA
gctcccccgccgtcgttcaatgagaatggataagaggctcgtgggattgacgtgagggggcagggatggctatatttctgggagcgaactccgggcgaatacgaagcgcttggatacgcatgcctgcagaaagaaggaagtcacgccagaggacacggagctgctccagaaggcgcagagcaatgtgagcgcatacaacagcgacgtcacctcgcgcatctgctacctgaaggtcgacagtctcgagactcaagtcgtctcgggcgagaactacaagttccacgtttccggttgcagcgtcaactcggacaaggagctgggcggctgtgccaatcagaattgcgagtcatccaagtacgacatcgtcatctactcgcagtcgtggaccaacacgctgaaggtgacgtcgattacgcccgccaacgctggtgccgcaggtaactcgtacatgtccatggcgacgcccaacgacgtcaagaactacacgaacgacgttggccagatccagtgggcgcaggtgccgctgaacgccgcgcttgacaagctcaagtcgtcccgtgagggtctgacatccgatgaggctgagaagcgtctggccgagtacggcccgaacaagctgccggaggagaaggtgaacaagctgacgctgttcctgggcttcatgtggaacccgctgtcgtgggccatggaggtggccgccgtgctgtcgattgtgctgctggattacgctgatttcgcgctgatcctgttcctgctgctgctaaacagatctcccgggtaccgagctcgtatccaagcgcttcgtattcgcccggagttcgctcccagaaatatagccatccctgccccctcacgtcaatcccacgagcctcttatccattctcattgaacgacggcgggggagc

SEQ ID NO: 8 - DNA, 28 bp, Artificial Sequence
  forward primer of the Prrn promoter; introducing a HindIII site
aaaaaagcttgctcccccgccgtcgttc

SEQ ID NO: 9 - DNA, 29 bp, Artificial Sequence
  reverse primer of the Prrn promoter; introducing an SphI site
aaaagcatgcgtatccaagcgcttcgtat

SEQ ID NO: 10 - DNA, 28 bp, Artificial Sequence
  forward primer of the Prrn promoter; introducing an EcoRI site
aaaagaattcgctcccccgccgtcgttc

SEQ ID NO: 11 - DNA, 29 bp, Artificial Sequence
  reverse primer of the Prrn promoter; introducing a SacI site
aaaagagctcgtatccaagcgcttcgtat

SEQ ID NO: 12 - DNA, 53 bp, Artificial Sequence
  forward primer of the Prrn promoter; introducing a PstI site
aaaactgcaggggtgggtggttttccacccacccgtatccaagcgcttcgtat

SEQ ID NO: 13 - DNA, 53 bp, Artificial Sequence
  reverse primer of the Prrn promoter; introducing a BamHI site
aaaaggatcccgcacgctccattaggagcgtgcggtatccaagcgcttcgtat

SEQ ID NO: 14 - DNA, 31 bp, Artificial Sequence
  forward primer of the ACT gene fragment; introducing an SbfI site
aaacctgcaggcacgaggtttttctgtctag

SEQ ID NO: 15 - DNA, 30 bp, Artificial Sequence
  reverse primer of the ACT gene fragment; introducing a SacI site
aaaagagctcatgtcatcccagttggtgat

SEQ ID NO: 16 - DNA, 32 bp, Artificial Sequence
  forward primer of the SHR gene fragment; introducing an SbfI site
aaaacctgcaggcaatgtcatccatcatgtcg

SEQ ID NO: 17 - DNA, 26 bp, Artificial Sequence
  reverse primer of the SHR gene fragment; introducing a SacI site
aaaagagctcgagcggccatgcaagc

SEQ ID NO: 18 - DNA, 36 bp, Artificial Sequence
  forward primer of the E. coli rrnB terminator; introducing a SacI site
aaaagagctcatcaaataaaacgaaaggctcagtcg

SEQ ID NO: 19 - DNA, 33 bp, Artificial Sequence
  reserve primer of the E. coli rrnB terminator; introducing an EcoRI site
aaaagaattcgtagaaacgcaaaaaggccatcc

SEQ ID NO: 20 - DNA, 28 bp, Artificial Sequence
  forward primer of the ACT+SHR fragment; introducing a SacI site
aaaagagctcgcacgaggtttttctgtc

SEQ ID NO: 21 - DNA, 25 bp, Artificial Sequence
  reverse primer of the ACT+SHR fragment; introducing a SmaI site
aaaacccgggagcggccatgcaagc

SEQ ID NO: 22 - DNA, 30 bp, Artificial Sequence
  forward primer of the ACT gene fragment; introducing an XbaI site
aaaatctagacacgaggtttttctgtctag

SEQ ID NO: 23 - DNA, 30 bp, Artificial Sequence
  forward primer of the ACT gene fragment; introducing an XhoI site
aaaactcgagcacgaggtttttctgtctag

SEQ ID NO: 24 - DNA, 29 bp, Artificial Sequence
  reverse primer of the ACT gene fragment; introducing a BglI site
aaaagatctatgtcatcccagttggtgat

SEQ ID NO: 25 - DNA, 30 bp, Artificial Sequence
  forward primer of the SHR gene fragment; introducing an XbaI site
aaaatctagacaatgtcatccatcatgtcg

SEQ ID NO: 26 - DNA, 30 bp, Artificial Sequence
  forward primer of the SHR gene fragment; introducing an XhoI site
aaaactcgagcaatgtcatccatcatgtcg

SEQ ID NO: 27 - DNA, 26 bp, Artificial Sequence
  reverse primer of the SHR gene fragment; introducing a BamHI site
aaaaggatccgagcggccatgcaagc

SEQ ID NO: 28 - DNA, 43 bp, Artificial Sequence
  forward primer of the ACT+SHR fragment; introducing the T7 promoter sequence; for in vitro dsRNA synthesis
taatacgactcactataggcctgcaggcacgaggtttttctgt

SEQ ID NO: 29 - DNA, 39 bp, Artificial Sequence
  reverse primer of the ACT+SHR fragment; introducing the T7 promoter sequence; for in vitro dsRNA synthesis
taatacgactcactataggggcccgggatccgatatgcc

SEQ ID NO: 30 - DNA, 40 bp, Artificial Sequence
  forward primer of the ACT fragment; introducing the T7 promoter sequence; for in vitro dsRNA synthesis
taatacgactcactataggatgtgtgacgacgatgtagcg

SEQ ID NO: 31 - DNA, 39 bp, Artificial Sequence
  reverse primer of the ACT fragment; introducing the T7 promoter sequence; for in vitro dsRNA synthesis
taatacgactcactataggttccatgtcatcccagttgg

SEQ ID NO: 32 - DNA, 41 bp, Artificial Sequence
  forward primer of the SHR fragment; introducing the T7 promoter sequence; for in vitro dsRNA synthesis
taatacgactcactatagggagtggccctgcaagccctcaa

SEQ ID NO: 33 - DNA, 39 bp, Artificial Sequence
  reverse primer of the SHR fragment; introducing the T7 promoter sequence; for in vitro dsRNA synthesis
taatacgactcactatagggcaatgtcatccatcatgtc

SEQ ID NO: 34 - DNA, 39 bp, Artificial Sequence
  forward primer of the gfp gene; introducing the T7 promoter sequence; for in vitro dsRNA synthesis
taatacgactcactataggaggacgacggcaactacaag

SEQ ID NO: 35 - DNA, 39 bp, Artificial Sequence
  reverse primer of the gfp gene; introducing the T7 promoter sequence; for in vitro dsRNA synthesis
taatacgactcactataggctgggtgctcaggtagtggt

SEQ ID NO: 36 - DNA, 20 bp, Artificial Sequence
  forward primer of ACT expression
ccagtcctcctcactgaagc

SEQ ID NO: 37 - DNA, 20 bp, Artificial Sequence
  reverse primer of ACT expression
acgaccagaagcgtacaagg

SEQ ID NO: 38 - DNA, 20 bp, Artificial Sequence
  forward primer of SHR expression
gatgatttggacgatgctga

SEQ ID NO: 39 - DNA, 20 bp, Artificial Sequence
  reverse primer of SHR expression
tagctggtttgactggcttg

SEQ ID NO: 40 - DNA, 20 bp, Artificial Sequence
  forward primer of RPS18 expression (as reference gene)
gcgggagaatgtacagagga

SEQ ID NO: 41 - DNA, 20 bp, Artificial Sequence
  reverse primer of RPS18 expression (as reference gene)
aagtcttcacggagcttgga

SEQ ID NO: 42 - DNA, 20 bp, Artificial Sequence
  forward primer of RPS4 expression (as reference gene)
cgtcaaagaaacgagcattg

SEQ ID NO: 43 - DNA, 20 bp, Artificial Sequence
  reverse primer of RPS4 expression (as reference gene)
tcgctgacactgtagggttg

SEQ ID NO: 44 - DNA, 12 bp, Artificial Sequence
  introducing SphI and PstI restriction sites
gcatgcctgcag

(19) United States
(12) Patent Application Publication
(10) Pub. No.: US 2017/0342429 A1
(43) Pub. Date: Nov. 30, 2017

(54) PLANT PROTECTION FROM A PEST OR PATHOGEN BY EXPRESSION OF DOUBLE-STRANDED RNAs IN THE PLASTID

(71) Applicant: Max-Planck-Gesellschaft zur Förderung der Wissenschaften e.V., Munich (DE)

(72) Inventors: Ralph Bock, Schwielowsee (DE); Jiang Zhang, Potsdam (DE); David G. Heckel, Jena (DE); Sher Afzal Khan, Jena (DE)

(73) Assignee: Max-Planck-Gesellschaft zur Förderung der Wissenschaften e.V., Munich (DE)

(21) Appl. No.: 15/537,410

(22) PCT Filed: Dec. 18, 2015
(86) PCT No.: PCT/EP2015/080570

§ 371 Date: Jun. 17, 2017

(30) Foreign Application Priority Data
Dec. 19, 2014 (EP) 14199415.2

(51) Int. Cl. C12N 15/82

(52) U.S. Cl. CPC C12N 15/8218; C12N 15/8285; C12N 15/8286; C12N 15/8214; C12N 15/8282; C12N 15/8279; C12N 2310/14

(57) ABSTRACT

The present invention lies in the field of plant protection, in particular in the field of controlling plant pests and pathogens that affect plants. The present invention relates to a plant comprising a plastid comprising a double-stranded RNA (dsRNA) capable of silencing at least one target gene of a pest of a plant or of an agent causing a disease of a plant. The present invention further relates to such a transplastomic plant, wherein said dsRNA comprises two (separate) complementary single-stranded RNA strands. The present invention further relates to a plastid as comprised in the plant of the invention and to a plant cell comprising said plastid. Moreover, the present invention relates to a method of producing a plant of the invention and to a method of controlling a pest of a plant or a plant disease-causing agent or of protecting a plant from said pest or agent. Furthermore, the present invention relates to the use of a dsRNA for controlling a pest of a plant or a plant disease-causing agent or for protecting a plant from said pest or agent.

The present invention lies in the field of plant protection, in particular in the field of plant protection from respective pests of plants and pathogens that affect plants. The present invention relates to a plant comprising a plastid comprising a double-stranded RNA (dsRNA) capable of silencing at least one target gene of a pest of a plant or of an agent causing a disease of a plant. The present invention further relates to such a transplastomic plant, wherein said dsRNA comprises two separate complementary single-stranded RNA strands. The present invention further relates to a plastid as comprised in the plant of the invention and to a plant cell comprising said plastid. Moreover, the present invention relates to a method of producing a plant of the invention and to a method of controlling a pest of a plant or a plant disease-causing agent or of protecting a plant from said pest or agent. Furthermore, the present invention relates to the use of a dsRNA for controlling a pest of a plant or a plant disease-causing agent or for protecting a plant from said pest or agent.

DsRNA fed to insects (or to other organisms) can be taken up by midgut cells and processed into small interfering RNAs (siRNAs) by Dicer endoribonuclease (1, 2, 3). If the sequence of the fed dsRNA is derived from an endogenous (insect) gene, specific gene silencing by RNA interference (RNAi) is induced (4, 3). By targeting essential (insect) genes, dsRNAs potentially can be developed into highly species-specific pesticides, e.g. insecticides (4). A number of recent studies have explored this approach for crop protection against pests by expressing dsRNAs targeted against pest (insect) genes in transgenic plants (1, 2, 5, 6, 7, 8).

The RNAi technology offers the possibility to choose target(s) from a vast number of genes. Growing problems with resistances of (insect) pests against chemical pesticides, e.g. insecticides and Bt toxins (17, 18, 19), therefore, make plant-produced dsRNAs a highly promising future strategy for plant protection. Moreover, it provides plant protection without chemicals and does not require synthesis of foreign proteins in the plant.

As insect target genes, for example, the ACT and SHR genes from the Colorado potato beetle (Leptinotarsa decemlineata; CPB), a notorious insect pest of potato and other Solanaceous plants, may be chosen, based on their high efficacy in inducing mortality in feeding assays with in vitro-synthesized dsRNAs (12, 3). ACT encodes β-actin, an essential cytoskeletal protein, and SHR encodes Shrub (also known as Vps32 or Snf7), an essential subunit of a protein complex involved in membrane remodeling for vesicle transport.

Protecting plants against viral pathogens by transforming them with transgenes encoding hairpin RNAs (hpRNAs), in particular loopless hpRNAs, has also been proposed (Smith, Nature 407 (2000), 319-320).

Multiple kind of RNA-based gene silencing constructs are known for gene silencing in plants. For example, gene fragments positioned between two oppositely orientated promoters were shown to make a transcriptional terminator unnecessary but nevertheless result in efficient gene silencing in plants (Yan, Plant Physiology 141 (2006), 1508-1518).

RNAi-mediated gene silencing approaches have also been used in technical fields other than the field of plant protection, for example in the field of human and animal health protection from respective parasites and pathogens (e.g., mosquitoes and viruses). For example, feeding or contacting these parasites/pathogens with silencing dsRNA expressed in the chloroplast of microalgae was applied in this context (WO 2012/054919; US 2013/0315883).

In particular, dsRNAs targeted against essential genes could trigger a lethal RNAi response upon uptake by pests or upon contact by pathogens. However, the application of this concept in plant protection has been hampered by the presence of an endogenous RNAi pathway in plants that effectively degrades dsRNAs into small interfering RNAs (siRNAs).

Although biological activity of dsRNA could be demonstrated, for example in that insect larvae feeding on the dsRNA-producing transgenic plants displayed impaired growth and development, complete protection of the plants and efficient killing of the insects was not achieved. Detailed investigations into the mechanism of RNAi induction by ingested RNAs revealed that a minimum length of dsRNAs of 50-60 base pairs (bp) is required for biological activity in particular if the plant pest is an insect (3). However, the presence of Dicer proteins in all plants and their essential roles in the biogenesis of endogenous small RNAs (9) prevent the stable accumulation of significant amounts of long dsRNA. 21 bp siRNAs, the major processing products of dsRNA cleavage by Dicer, showed either only small effects (10) or no gene silencing activity at all in artificial diet bioassays with insects (3), indicating that the rapid turnover of dsRNAs in the plant limits the efficacy of transgenic RNAi-based anti-pest/pathogen strategies.

Moreover, RNA-dependent RNA polymerase (RdRP) genes are absent from the genomes of, for example, insects (16). Therefore, silencing signals are not amplified at the RNA level and RNAi effects remain restricted to those cells that have taken up (or produced) silencing-inducing dsRNAs. Consequently, a continuous input of dsRNAs is required for efficient gene silencing by RNAi. Due to the low stability of dsRNAs expressed from the nuclear genome and their efficient degradation by Dicer endoribonucleases, complete protection of plants from plant pests and plant pathogens has not been accomplished (1, 2).

Attempts have been made to address this deficiency, for example by applying silencing dsRNA with stabilizing features like mismatches and stem-loop structures which render the dsRNA more resistant against plant Dicer endoribonucleases (WO 2007/011479; U.S. Ser. No. 11/453,155).

However, a simple and effective approach which exploits the whole potential of the RNAi technology in plant protection has not yet been achieved.

Thus, the technical problem underlying the present invention is the provision of reliable and improved means and methods for an effective and moreover complete plant protection from plant pests and from plant pathogens.

The technical problem is solved by the provision of the embodiments as prescribed herein and, in particular, as characterized in the claims.

Accordingly, the present invention relates to a plant comprising a plastid comprising a dsRNA capable of silencing at least one target gene of a pest of a plant (also referred to herein as a “plant pest”) or of an agent causing a disease of a plant (also referred to herein as a “disease-causing agent” or “plant pathogen”), wherein said dsRNA comprises two complementary single-stranded RNA strands. Such a plant, in particular if it has been genetically engineered so as to comprise said plastid, is also termed a “transplastomic” plant.

The present invention solves the above identified technical problem since, as documented herein below and in the appended examples, it was surprisingly found that plastids of plant cells (in particular chloroplasts) are capable of stably accumulating high amounts of long dsRNAs, in which case silencing dsRNA expression from the plastids' genome could provide much better protection against plant pests and plant pathogens as compared to dsRNA expression from the nuclear genome. In particular, it was surprising that a sound and even complete plant protection was achieved by expressing silencing dsRNA in the plastid.

More particular, it was found in the context of the invention that large amounts of long dsRNAs targeted against plant-feeding insect genes and fungal plant pathogen genes can be produced in the chloroplast and provide full protection of the respective plants from the respective insects (for example of potato plants from the CPB, a notorious agricultural pest) and from the respective fungus (for example of potato plants from the oomycete Phytophthora infestans, the causative agent of potato blight), respectively.

Specifically, transplastomic potato plants producing dsRNAs targeted against the 3-actin gene of CPB were shown to be protected from herbivory by CPB and cause complete mortality to CPB larvae.

Likewise, transplastomic potato plants producing dsRNA targeted against the EPIC2B and/or PnPMA1 gene(s) of Phytophthora infestans, the causative agent of potato blight, were shown to be protected from attack/damage caused by said pathogen.

Furthermore, especially when expressed from the plastid's genome, e.g. from the chloroplast's genome, dsRNAs were shown to accumulate to up to 0.4% of the total cellular RNA. Hence, the dsRNA may accumulate to, e.g., at least 0.05%, at least 0.1%, 0.2%, 0.3% or 0.4% of the total cellular RNA (the higher values are preferred).

Without being bound by theory, the advantageous effects underlying the invention may, at least in part, be due to the absence of an efficient dsRNA-degrading mechanism/RNAi machinery in plastids.

The data reported herein underscore the importance of producing large amounts of long dsRNAs to achieve efficient plant protection. The invention complies with this need and offers a highly efficient strategy for plant protection, in particular crop protection, without chemicals. The findings that plastids can be genetically engineered to stably accumulate large amounts of dsRNAs and that, in this way, major agricultural pests like the CPB can be fully controlled, remove the major hurdle on the way to exploiting RNAi for efficient plant protection in the field (16).

One advantage of the means and methods of the invention results from the findings that all transplastomic lines displayed no visible phenotype and were indistinguishable from wild-type plants, both under in vitro culture conditions and upon growth in the greenhouse. This indicates that dsRNA expression in the chloroplast is phenotypically neutral.

A further surprising finding in the context of the invention was that transplastomic plants with plastids expressing silencing dsRNA comprising two separate complementary single-stranded RNA strands provide for extraordinary good results in terms of high amounts and stable accumulation of long dsRNAs.

Accordingly, in a preferred embodiment, the plant of the invention comprises a plastid comprising a dsRNA capable of silencing at least one target gene of a plant pest or of a plant pathogen, wherein said dsRNA comprises two separate complementary single-stranded RNA strands.

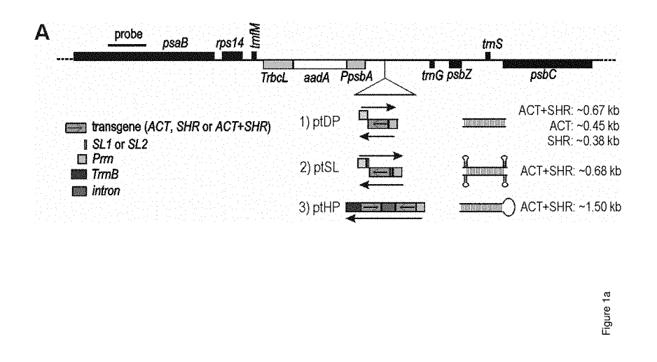

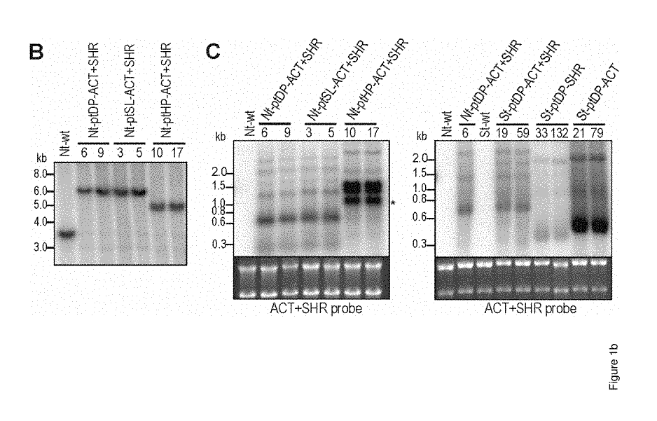

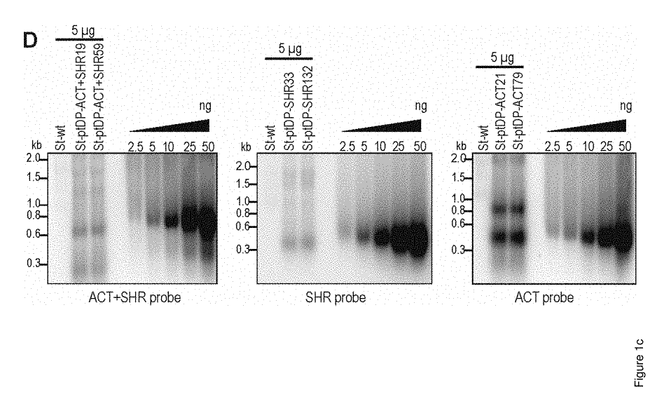

“Separate” in the context of the invention and, in particular, of this preferred embodiment means that the two RNA strands are not covalently bound to each other. For example, they are not linked/connected by a loop of a single-stranded RNA strand. The two “separate” RNA strands may, however, be connected via hydrogen bonds due to (an) hybridization event(s), preferably over a length of 50 or more consecutive base pairs. Such two RNA strands which are bound to each other are still considered “separate” in accordance with the invention (as long as they are not covalently bound to each other). The two “separate” RNA strands may be transcribed from two transgene copies arranged as an inverted repeat (see, for example, FIG. 1A; “ptHP”). The two “Separate” RNA strands may preferably be generated by transcription from a template by two convergent promoters (see, for example, FIG. 1A; “ptDP” or “ptSL”).

Examples of preferred approaches to express high amounts of long dsRNA in the plastid of a respective plant in accordance with this preferred embodiment (“separate” RNA strands) are also referred to herein as the “ptDP” and “ptSL” approaches; the respective constructs encoding the dsRNA are referred to as “ptDP” constructs and “ptSL” constructs, respectively.

In the context of the “ptDP” approach and the “ptDP” constructs, and in the context of the “ptSL” approach, and the “ptSL” contstructs, the dsRNA is generated by transcription from two convergent promoters. In the “ptSL” approach, one or each strand of the dsRNA is flanked by sequences forming stem loop-type secondary structures, or other/further stabilizing elements. Such elements are known to increase RNA stability in plastids (11).

Hence, in the context of this preferred embodiment, the dsRNA may be expressed by transcription from a nucleotide sequence (for example DNA) flanked by two convergent promoters.

In principle, the dsRNA to be employed in accordance with the invention may also comprise two complementary single-stranded RNA strands which are not “separate”, i.e. which are covalently bound to each other. Such dsRNA may be formed by one single RNA strand via (an) hybridization event(s) of two complementary regions (preferably over a length of 50 or more consecutive base pairs) which are comprised in this single RNA strand. Typically, the resulting dsRNA may form a hairpin/stem-loop structure (hpRNA). Such a structure may comprise a loop or may be a loopless hairpin/stem-loop structure.

An example of an approach to express “non-separate” dsRNA strands in the plastids of the plant of the invention is also referred to herein as the “ptHP” approach; the respective constructs are referred to as the “ptHP” constructs. In the context of the “ptHP” approach hpRNA may be produced by transcription of two transgene copies arranged as inverted repeat (see, for example, FIG. 1A, 1)).

As to the “ptDP”, “ptSL” and “ptHP” approaches and the respective constructs, illustrative reference is also made to FIG. 1A 1), 2) and 3), respectively, and to the respective examples.

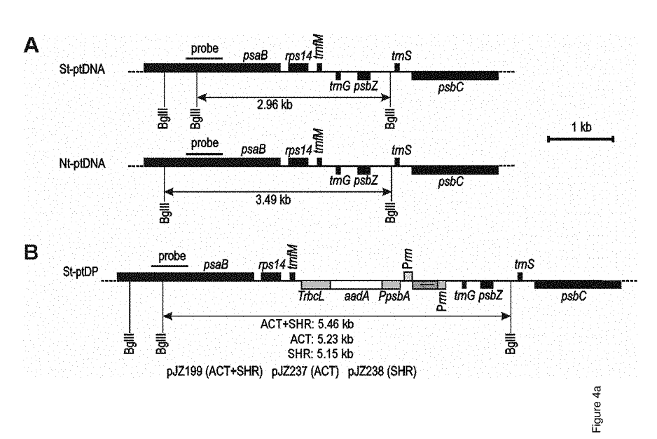

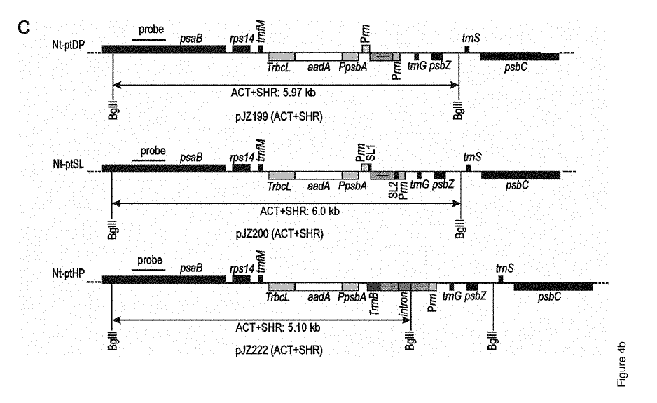

Another surprising finding in the context of the invention was that ACT dsRNA was slightly more effective than the SHR dsRNA, whereas the ACT+SHR dsRNA was significantly less effective than either the ACT or SHR dsRNAs (see, for example, FIG. 5). This indicates that some target genes are more effective than others and that targeting two (or more) insect genes (or two (or more) other plant pest/pathogen genes) with the same dsRNA may not necessarily enhance anti-plant pest/pathogen activity (e.g. insecticidal activity).

Hence, in a preferred embodiment, the dsRNA to be employed in accordance with the invention targets only one gene of a plant pest or of a plant pathogen. However, in principle, also 2, 3, 4, 5 or even more genes may be targeted (the higher amounts are less preferred). In particular, 2, 3, 4, 5 or even more genes may be targeted by expression of the respective dsRNAs from separate nucleotide sequences (e.g. separate recombinant DNA constructs). This could be achieved by, for example, introducing 2, 3, 4, 5 or even more “ptDP”, “ptSL” or “ptHP” cassettes into the plastid's genome, rather than expressing them from a fusion gene.

Targeting more than one gene like this could even result in a more efficient control of the respective plant pest or plant pathogen.

Likewise, in particular in case the plant pest is an insect (like the CPB), targeting ACT is preferred.

However, in principle, the skilled person may choose any target gene (or two (or more) target genes) of a plant pest or of a plant pathogen which, when being silenced, results in a significant control of the respective plant pest or plant pathogen and of a significant/sufficient protection of a plant from said pest or pathogen, respectively. Non limiting examples of such target gene(s) and the respective plant pest/plant pathogen are given herein elsewhere.

Another advantage of the invention is that, depending on regulatory elements, the expression of most plastid genes is drastically down-regulated in, for example, non-photosynthetic tissues (c.f. 14, 15). This provides for the possibility to prevent dsRNA production in, for example, non-photosynthetically active plant tissue or parts of a plant (like, for example, tubers, stems, roots, underground shoots, fruits, seeds, etc.) where the accumulation of transgene-derived RNA may be unnecessary and/or probably undesired by the consumer. Comparative analyses of dsRNA accumulation in leaves and tubers revealed that, depending on the regulatory elements, dsRNA levels in tubers are nearly undetectably low (see, for example, FIG. 1F).

Hence, in a preferred embodiment, the plastid which comprises the dsRNA to be employed in accordance with the invention is a chloroplast. However, for example by choosing appropriate expression signals, it is also possible to express plastid transgenes encoding the dsRNA to high levels in non-green tissues, i.e. in other types of plastids (cf. Zhang Plant J. 72 (2012) 115-128; Caroca Plant J. 73 (2013) 368-379).

Thus, also other plastids may comprise the dsRNA in accordance with the invention. Examples of such plastids are plastids contained in the phloem (P-plastids), pro-plastids, chromoplasts, leucoplasts (e.g. amyloplasts, proteinoplasts, elaioplasts) and gerontoplasts.

The skilled person is readily able to provide plants which comprise/express the dsRNA to be employed in accordance with the invention in certain plastids (e.g. in chloroplasts) and not to comprise/express the dsRNA in other plastids (e.g. amyloplasts), as the case may be. For example, such a selective expression/production of the dsRNA in the respective plastids can readily be achieved by the choice of, for example, (a) respective suitable element(s) like (a) promoter(s) and/or (a) signaling sequence(s).

A particular but non-limiting example of a promoter which may be used to express the dsRNA in the plastid (e.g. in the chloroplast) is the Prrn promoter (or two convergent Prrn promoters). Other suitable promoters are, for example, the plastid psbA, psbD, rbcL and rp132 promoters, or two convergent psbA, psbD, rbcL and rp132 promoters, respectively (see, for example, Staub (1993) EMBO J. 12, 601-606; Allison (1995) EMBO J. 14, 3721-3730; Eibl (1999) Plant J. 19, 333-345). In principle, heterologous promoters from other organisms (e.g. bacteria and phages) may also be used (see, for example, Newell (2003) Transgenic Res. 12, 631-634).

Generally, the nucleotide sequence (e.g. the recombinant DNA construct) which encodes the dsRNA may include a promoter operably linked to the transcribable nucleotide sequence. In various embodiments, the promoter is selected from the group consisting of a constitutive promoter, a spatially specific promoter, a temporally specific promoter, a developmentally specific promoter, and an inducible promoter.

Non-constitutive promoters suitable for use with the recombinant DNA constructs of the invention include spatially specific promoters, temporally specific promoters, and inducible promoters. Spatially specific promoters can include cell-, tissue-, or organ-specific promoters. Temporally specific promoters can include promoters that tend to promote expression during certain developmental stages in a plant's growth cycle, or during different times of day or night, or at different seasons in a year. Inducible promoters include promoters induced by chemicals or by environmental conditions such as, but not limited to, biotic or abiotic stress (e.g., water deficit or drought, heat, cold, high or low nutrient or salt levels, high or low light levels, or pest or pathogen infection). An expression-specific promoter can also include promoters that are generally constitutively expressed but at differing degrees or “strengths” of expression, including promoters commonly regarded as “strong promoters” or as “weak promoters”.

In accordance with the invention, a dsRNA is capable of silencing a target gene when it induces an RNAi response as to the respective target gene. Usually, this occurs if the dsRNA shares a substantial sequence identity with at least a (coding) part of the respective target gene, e.g. at least 60% sequence identity over a certain length (e.g. over at least 50 contiguous nucleotides/bps). Such dsRNAs are also referred to herein as “long” dsRNAs.

In principle, the dsRNA may correspond to any part of the target gene, for example to (a) regulatory sequence(s), like the promoter, signaling or targeting sequence(s), or to the coding sequence, i.e. (parts of) the sequence of the mRNA. It is particularly preferred that the dsRNA corresponds to (parts of) the mRNA of the target gene. “Corresponding to” in this context means showing substantial sequence similarity or, preferably, sequence identity (for example as described herein elsewhere) over a certain length (for example as described herein elsewhere).

The target gene can be a translatable (coding) sequence (preferred), or can be non-coding sequence (such as non-coding regulatory sequence), or both. Non-limiting examples of a target gene include non-translatable (non-coding) sequence, such as, but not limited to, 5′ untranslated regions, promoters, enhancers, or other non-coding transcriptional regions, 3′ untranslated regions, terminators, and introns. Target genes include genes encoding microRNAs, small interfering RNAs, RNA components of ribosomes or ribozymes, small nucleolar RNAs, and other non-coding RNAs (see, for example, non-coding RNA sequences provided publicly at rfam.wustl.edu; Erdmann et al. (2001) Nucleic Acids Res., 29:189-193; Gottesman (2005) Trends Genet., 21:399-404; Griffiths-Jones et al. (2005) Nucleic Acids Res., 33:121-124). Target genes can also include a translatable (coding) sequence for genes, for example encoding transcription factors and genes encoding enzymes involved in the biosynthesis or catabolism of molecules of interest (such as, but not limited to, amino acids, fatty acids and other lipids, sugars and other carbohydrates, biological polymers, and secondary metabolites including alkaloids, terpenoids, polyketides, non-ribosomal peptides, and secondary metabolites of mixed biosynthetic origin).

As mentioned, in the context of the invention, the term “long” dsRNA means any length of a dsRNA which leads to a (considerable) silencing of the respective target gene. In particular, “long” means that the dsRNA is at least 50 bp in length. Hence, in one embodiment, the dsRNA to be employed in the context of the invention is at least 50 bp in length. More particular, the dsRNA may be about 50-1000, 100-800, 150-650, 160-500, 170-400, 180-300 bps in length. In principle, the smaller ranges are preferred. In a more specific embodiment, the dsRNA is about 180-250 bps in length. In principle, however, the length of the dsRNA is not limiting, as long as it leads to a (considerable) silencing of the respective target gene. The skilled person is readily in the position to choose the (respective length(s)). In principle, “long” dsRNAs to be employed in the context of the invention is envisaged to be longer than the major processing products of dsRNA cleavage by Dicer, i.e. longer than siRNAs of about 21 bp in length.

In principle, “(considerable) silencing” in the context of the invention means that the expression of the target gene is reduced so that the respective plant pest or plant pathogen is impaired in any manner, in particular impaired so that its damage/harm to the plant (for example the extent of fed leaf feed) is reduced. In this context, functioning, growth, development, infectivity, mobility and/or reproduction of the plant pest/pathogen may be impaired. It is preferred, that the expression of the target gene is reduced to an extent which is lethal to the plant pest/pathogen. For example, the expression of the target gene may, in accordance with the invention, be reduced by at least 10%, 20%, 30%, 40%, 50%, 60%, 70%, 80%, 90, 95% or even 100% (the higher values are preferred).

In particular, it is envisaged that the above ranges of length of the dsRNA correspond to that part of the dsRNA which represents the respective nucleotide sequence stretch of the target gene to be silenced. In particular, “representing” means in this context that the sense strand of the dsRNA is similar or, preferably, identical to the sense strand of the respective target gene and/or to the respective nucleotide sequence stretch of an mRNA described from the target gene. For example, the sense strand of the dsRNA to be employed in the context of the invention may be at least 60%, 70%, 80%, 90%, 95%, 98%, 99% or 100% identical to the sense strand of the respective target gene and/or to an mRNA transcribed from the target gene, wherein the higher values are preferred. It is preferred that these identity values are seen with respect to the above-mentioned ranges of length. For example, the sense strand of the dsRNA may be at least 60% identical to (an mRNA transcribed from) a nucleotide sequence of at least 50 contiguous nucleotides/bps of the target gene, etc. What has been said above with respect to the ranges of length of the dsRNA also applies here, mutatis mutandis.

Besides the double-stranded part (for example in any of the above-mentioned lengths), the dsRNA to employed in context of the invention may comprise further components. There may be one or more single-stranded overhang nucleotide sequence(s) (e.g. DNA or (preferably) RNA) and/or one or more further double-stranded nucleotide sequence stretche(s) (e.g. DNA or (preferably) RNA). The nucleotide sequence of such a further component may not necessarily be similar (or identical) to a nucleotide sequence of the target gene.

In particular, the dsRNA, and at least one of its (separate) RNA strands, respectively, may comprise at least one of such further component(s). More particular, the dsRNA, and at least one of its (separate) RNA strands, respectively, may comprise at least one stabilizing feature. Such one or more stabilizing feature(s) may confer the dsRNA with an improved resistance to RNases, in particular to plastid RNases. Such stabilizing features are well known in the art and are, for example, described in WO 2007/011497, for example, in FIG. 1 and paragraph [0027] thereof. For example, such stabilizing features are DNA or (preferably) RNA sequences. The nucleotide sequence(s) of the stabilizing feature(s) to be employed in the context of the invention may differ from these stabilizing features and from further/other such stabilizing features known in the art, for example depending on the particular gene to be targeted by the respective dsRNA. The person skilled in the art is readily able to choose suitable stabilizing features.

A particular stabilizing feature may be a nucleotide sequence stemloop/hairpin structure (hp structure; e.g. DNA or (preferably) RNA). Such a hp structure may comprise a double-stranded nucleotide stretch (RNA stretch) of about 2-20 bps, 2-10 bps, 4-10 bps, 4-8 bps, or at least 2 bps, 3 bps, 4 bps, 5 bps, 6 bps, 7 bps, 8 bps, 9 bps or 10 bps. Moreover, such a bp structure may (further) comprise a single-stranded nucleotide stretch (the “loop”) of 0-20, 0-10, 1-20, 1-10, 2-20, 2-10, or at least 1, 2, 3, 4, 5, 6, 7, 8, 9 or 10 nucleotides.

In one specific embodiment of the invention, each of the two (separate) RNA strands may comprise a stabilizing feature, e.g. a hp structure, at its 5′- or 3′-end, preferably at its 5′- and 3′-end. An approach where such a dsRNA is employed is also referred to herein as the “ptSL” approach.

Although the dsRNA to be employed in the context of the invention may comprise (a) further component(s) (e.g. (a) stabilizing feature(s)), it is envisaged as a specific and preferred aspect of the invention that the dsRNA is free of such components (e.g. free from such stabilizing features), i.e. that the dsRNA merely consists of the two complementary single-stranded RNA strands, more preferably of the two separate complementary single-stranded RNA strands. An approach where such a dsRNA is employed is also referred herein as the “ptDP” approach. Such an approach and the respective expression constructs, plastids, plants, etc. provides for the further advantage that it is simple but comparably or even more effective, for example as compared to the “ptSL” and “ptHP” approaches.

The plant of the invention, and/or the plastid(s) comprised in the plant of the invention, may be genetically engineered so that the plastid(s) comprise a nucleotide sequence (e.g. recombinant DNA construct) encoding the dsRNA to be employed in accordance with the invention. The dsRNA may then be transcribed/expressed from said nucleotide sequence. Illustrative but non-limiting examples of such nucleotide sequences are the “ptDP”, “ptSL” and “ptHP” constructs described herein (cf. FIGS. 1A and 4).

Various embodiments of the nucleotide sequence encoding the dsRNA (e.g. recombinant DNA construct) as employed in the context of invention include, in addition to the transcribable nucleotide sequence (coding for the dsRNA), one or more of the following elements:
(a) a plastid promoter or a promoter from a heterologous source organism that is active in plastids (e.g. from a bacterium or phage);
(b) a ribozyme flanking the transcribable DNA;
(c) an intron that is embedded in the transcribable DNA;
(d) DNA that transcribes to an RNA aptamer capable of binding to a ligand;
(e) DNA that transcribes to an RNA aptamer capable of binding to a ligand and DNA that transcribes to regulatory RNA capable of regulating expression of a target sequence, wherein the regulation is dependent on the conformation of the regulatory RNA, and the conformation of the regulatory RNA is allosterically affected by the binding state of the RNA aptamer; and
(f) at least one gene expression element.

These elements are, for example, described in more detail herein elsewhere and in WO 2007/011479.

In one aspect of the invention, recombinant DNA constructs are used, wherein the target gene is exogenous to the plant in which the construct is to be transcribed, but endogenous to a pest or pathogen (e.g., fungi and invertebrates such as insects, nematodes, and molluscs) of the plant. The target gene can include multiple target genes, or multiple segments of one or more genes; one target gene per expression cassette, however, is preferred. In one preferred embodiment, the target gene or genes is a gene or genes of an invertebrate pest or pathogen of the plant. These nucleotide sequences (recombinant DNA constructs) are particularly useful in providing transgenic plants having resistance to one or more plant pests or plant pathogens, for example, resistance to a nematode such as soybean cyst nematode or root knot nematode or to a pest insect.

The nucleotide sequence encoding the dsRNA may be introduced into the plastid's genome. The dsRNA may be transcribed/expressed from the plastid's genome, for example from the mentioned introduced nucleotide sequence. Again, the “ptDP”, “ptSL” and “ptHP” constructs described herein are respective non-limiting examples of such nucleotide sequences.

The dsRNA may be transcribed/expressed directly in the plastid, for example from the mentioned nucleotide sequence and/or from the plastids genome, respectively.

Means and methods to genetically engineer a plant and/or a plastid(s) comprised therein (so that the plastid(s) comprise (e.g. express/produce) the dsRNA in accordance with the invention) are known in the art and are, for example, described in references 13 and 28. An example of such a method is biolistic transformation (particle bombardment), for example with gold particles coated with the nucleotide sequence (e.g. recombinant DNA construct) encoding the dsRNA. The respective means may, for example be a PDS1000/He particle delivery system, for example equipped with a Hepta adaptor (BioRad, Hercules, Calif., USA).

Where a nucleotide sequence (recombinant DNA construct) is used to produce a transgenic, i.e. transplastomic, plant cell or transgenic, i.e. transplastomic, plant of this invention, genetic engineering and transformation, respectively, can include any of the well-known and demonstrated methods and compositions. Suitable methods for plant transformation include virtually any method by which DNA can be introduced into a cell, in particular into a plastid, such as by direct delivery of DNA (e.g., by PEG-mediated transformation of protoplasts, by electroporation, by agitation with silicon carbide fibers, and by acceleration of DNA coated particles), by Agrobacterium-mediated transformation, by viral or other vectors, etc. One preferred method of plant transformation is microprojectile bombardment, for example, as illustrated in U.S. Pat. Nos. 5,015,580 (soy), 5,550,318 (maize), 5,538,880 (maize), 6,153,812 (wheat), 6,160,208 (maize), 6,288,312 (rice) and 6,399,861 (maize), and 6,403,865 (maize), all of which are incorporated by reference.

In principle, the plant pest or disease-causing agent/plant pathogen in accordance with the invention may be any organism which affects a plant.

In particular, the plant pest or plant pathogen in accordance with the invention is envisaged to be an organism that causes considerable undesired damage to useful plants, in particular to agricultural plants like crop, plants, and/or that is known (by the skilled person) to cause such an undesired damage. “Affecting” a plant in accordance with the invention particularly means that the plant pest/pathogen causes considerable and undesired damage to the plant. In particular, it means that the plant pest/pathogen feeds on the plant. More particular, especially in case of a plant pest, it means that the plant pest eats (parts of) the plant, for example eats (a) certain tissue(s) of the plant (e.g. leave, stem and/or root tissue) or sucks (a) certain sap of the plant (e.g. phloem sap).

The meaning of “plant pest” and “plant pathogen” in accordance with the invention is particularly envisaged not to encompass animal/human pests or animal/human pathogens, even though they may, for example at a certain developmental stage, eat plants and feed on plants, respectively.

Depending on the pest or disease-causing agent, affecting a plant may more particularly mean, for example, that the pest feeds on the plant, in particular so that it eats the (genetically engineered) plastids of the plant comprising the dsRNA as employed in accordance with the invention. In other words, the pest or disease-causing agent is envisaged to feed on/eat those parts of a plant which comprises the (genetically engineered) plastids like, for example (parts of) the leaves. It is required that the plant pest or plant disease-causing agent is at least contacted with and/or takes up the dsRNA to be employed in the invention, i.e. the dsRNA comprised in the plastids of the invention, so that an RNAi response with respect to the target gene takes place.

Examples of respective plants, plant pests, plant pathogens and a selection of respective target genes are given herein elsewhere and are, for example described in WO 2007/011479.

In principle, the term “plant pest” encompasses any developmental stage of a respective organism, e.g. a larva/larval, a nymph/nymphs, a pupa/pupae and an adult/adults. In particular, the plant pest is envisaged to be an invertebrate plant pest.

More particular, the plant pest may be selected from the group consisting of:

(i) an arthropod;

(ii) a nematode; and

(iii) a mollusk like, for example, a snail or a slug.

The arthropod may be an insect or a mite. In principle, any herbivorous or plant sap-sucking plant pest, e.g. insect, mite, nematode or mollusk, is envisaged to be a pest in accordance with the invention.

The insect may, in a particularly preferred embodiment, be a CPB (Leptinotarsa decemlineata), including any juvenile stage of said beetle.

Plant pest invertebrates include, but are not limited to, pest nematodes, pest mollusks (slugs and snails), and pest insects. Plant pathogens of interest include fungi. See also G. N. Agrios, “Plant Pathology” (Fourth Edition), Academic Press, San Diego, 1997, 635 pp., for descriptions of fungi, nematodes, all of which are plant pests or pathogens of interest. See also the continually updated compilation of plant pests and pathogens and the diseases caused by such on the American Phytopathological Society's “Common Names of Plant Diseases”, compiled by the Committee on Standardization of Common Names for Plant Diseases of The American Phytopathological Society, 1978-2005, available online at www.apsnet.org/online/common/top.asp.

Non-limiting examples of invertebrate pests include cyst nematodes Heterodera spp. especially soybean cyst nematode Heterodera glycines, root knot nematodes Meloidogyne spp., lance nematodes Hoplolaimus spp., stunt nematodes Tylenchorhynchus spp., spiral nematodes Helicotylenchus spp., lesion nematodes Pratylenchus spp., ring nematodes Criconema spp., foliar nematodes Aphelenchus spp. or Aphelenchoides spp., corn rootworms, Lygus spp., aphids and similar sap-sucking insects such as phylloxera (Daktulosphaira vitifoliae), corn borers, cutworms, armyworms, leafhoppers, Japanese beetles, grasshoppers, and other pest coleopterans, dipterans, and lepidopterans. Specific examples of invertebrate pests include pests capable of infesting the root systems of crop plants, e.g., northern corn rootworm (Diabrotica barberi), southern corn rootworm (Diabrotica undecimpunctata), Western corn rootworm (Diabrotica virgifera), corn root aphid (Anuraphis maidiradicis), black cutworm (Agrotis ipsilon), glassy cutworm (Crymodes devastator), dingy cutworm (Feltia ducens), claybacked cutworm (Agrotis gladiaria), wireworm (Melanotus spp., Aeolus mellillus), wheat wireworm (Aeolus mancus), sand wireworm (Horistonotus uhlerii), maize billbug (Sphenophorus maidis), timothy billbug (Sphenophorus zeae), bluegrass billbug (Sphenophorus parvulus), southern corn billbug (Sphenophorus callosus), white grubs (Phyllophaga spp.), seedcorn maggot (Delia platura), grape colaspis (Colaspis brunnea), seedcorn beetle (Stenolophus lecontei), and slender seedcorn beetle (Clivinia impressifrons), as well as the parasitic nematodes listed in Table 6 of U.S. Pat. No. 6,194,636, which is incorporated in its entirety by reference herein.

The plant pathogen may, in particular be a eukaryotic plant pathogen. This includes for example, a fungal pathogen, in particular a phytopathogenic fungus.

A preferred but non-limiting example of a fungal pathogen is Phytophthora infestans (an oomycete). Phytophthora infestans is known to be the causative agent of potato blight.

Non-limiting examples of fungal plant pathogens of particular interest also include, e.g., the fungi that cause powdery mildew, rust, leaf spot and blight, damping-off, root rot, crown rot, cotton boll rot, stem canker, twig canker, vascular wilt, smut, or mold, including, but not limited to, Fusarium spp., Phakospora spp., Rhizoctonia spp., Aspergillus spp., Gibberella spp., Pyricularia spp., Alternaria spp., and Phytophthora spp. Specific examples of fungal plant pathogens include Phakospora pachirhizi (Asian soy rust), Puccinia sorghi (corn common rust), Puccinia polysora (corn Southern rust), Fusarium oxysporum and other Fusarium spp., Alternaria spp., Penicillium spp., Pythium aphanidermatum and other Pythium spp., Rhizoctonia solani, Exserohilum turcicum (Northern corn leaf blight), Bipolaris maydis (Southern corn leaf blight), Ustilago maydis (corn smut), Fusarium graminearum (Gibberella zeae), Fusarium verticilliodes (Gibberella moniliformis), F. proliferatum (G. fujikuroi var. intermedia), F. sub glutinous (G. subglutinans), Diplodia maydis, Sporisorium holci-sorghi, Colletotrichum graminicola, Setosphaeria turcica, Aureobasidium zeae, Phytophthora infestans, Phytophthora sojae, Sclerotinia sclerotiorum, and the numerous fungal species provided in Tables 4 and 5 of U.S. Pat. No. 6,194,636, which is incorporated in its entirety by reference herein.

Particular, but not-limiting examples of plant pests and plant pathogens, the respective plants and the respective target genes in accordance with the invention are well known in the art and are, for example, described in WO 2007/011479, The 2014 North Carolina Agricultural Chemicals Manual, published by the North Carolina Cooperative Extension Service, College of Agriculture and Life Sciences, N.C. State University, Raleigh, N.C., http://en.wikipedia.orG/wiki/Category:Agricultural_pest_insects, Entomology and Pest Management (4th Edition)—May 30, 2001 by Larry P. Pedigo ISBN-13: 978-0130195678 ISBN-10: 0130195677, Plant-Parasitic Nematodes: A Pictorial Key to Genera (Comstock Books) Hardcover—Feb. 8, 1996 by William Mai (Author), ISBN-13: 978-0801431166 ISBN-10: 0801431166 Edition: 5^{th}, and Pest Slugs and Snails: Biology and Control Paperback—Dec. 7, 2011 by D. Godan (Author), S. Gruber (Translator)ISBN-13: 978-3642687990 ISBN-10: 3642687997 Edition: Softcover reprint of the original 1st ed. 1983.

In particular, WO 2007/011479 exemplifies plant pests and plant pathogens, in particular plant pest invertebrates and fungal plant pathogens (see, e.g., paragraphs [0053], [0054] and [0057], and the respective plants (see, e.g., paragraph [00115]) and target genes (see, e.g. paragraphs [0050] to [0052] and [0058] to [0068]).

The transgenic, i.e. transplastomic, plant cell or transgenic, i.e. transplastomic, plant of the invention can be any suitable plant cell or plant of interest, as long as its plastid(s) comprise the dsRNA in accordance with the invention. Both transiently transformed and stably transformed plant cells are encompassed by this invention. Stably transformed transgenic plants are particularly preferred. In many preferred embodiments, the transgenic plant is a fertile transgenic plant from which seed can be harvested, and the invention further claims transgenic seeds of such transgenic plants, wherein the seeds preferably also contain the recombinant construct of this invention.

It is particularly envisaged that, in accordance with the present invention, the meaning of the term “plant(s)” excludes algae. Algae in this context particularly means Euglenophyta, Crysophyta, Pyrrophyta, Chlorophyta, Phaeophyta or Rhodophyta. A particular alga which is not envisaged to be a plant in accordance with the invention is a microalga, in particular a Chlamydomonas alga or a Chlamydomonas-like alga. It is acknowledged in the art that, for example, a Chlamydomonas alga is a “hybrid organism”, somewhere between animals and plants. This has also been confirmed by sequencing of its genome (see, e.g., Merchant, (2007), Science 318, 245-251). In one aspect, it is particularly envisaged that the plant of the invention is not a microalga being a member of one of the following divisions: Chlorophyta, Cyanophyta (Cyanobacteria), and Heterokontophyta. In certain aspects, the plant is not a microalga of one of the following classes: Chlorophyceae, Bacillariophyceae, Eustigmatophyceae, and Chrysophyceae. In certain aspects, the plant is not a mircoalga of one of the following genera: Chlamydomonas, Nannochloropsis, Chlorella, Dunaliella, Scenedesmus, Selenastrum, Oscillatoria, Phormidium, Spirulina, Amphora, and Ochromonas. In one aspect the plant is not a microalga of the genus Chlamydomonas. In one aspect, the plant of the invention is not a microalga of the following species: Chlamydomas perigranulata, Chlamydomonas moewusii. Chlamydomonas reinhardtii and Chlamydomonas sp.

In a preferred embodiment, the plant of the invention is a vascular plant, more preferably a spermatophyte.

The transgenic plant cells or transgenic plants of the invention, comprising the (genetically engineered) plastid of the invention, can be obtained by use of any appropriate transient or stable, integrative or non-integrative transformation method known in the art or presently disclosed. The respective nucleotide sequences (e.g. recombinant DNA constructs) can be transcribed in any plant cell or tissue or in a whole plant of any developmental stage, in particular in the plastid(s) thereof.

Transplastomic plants of the invention can be derived from any monocot or dicot plant, such as, but not limited to, plants of commercial or agricultural interest, such as crop plants (especially crop plants used for human food or animal feed), wood- or pulp-producing trees, vegetable plants, fruit plants, and ornamental plants. Non-limiting examples of plants of interest include grain crop plants (such as wheat, oat, barley, maize, rye, triticale, rice, millet, sorghum, quinoa, amaranth, and buckwheat); forage crop plants (such as forage grasses and forage dicots including alfalfa, vetch, clover, and the like); oilseed crop plants (such as cotton, safflower, sunflower, soybean, canola, rapeseed, flax, peanuts, and oil palm); tree nuts (such as walnut, cashew, hazelnut, pecan, almond, and the like); sugarcane, coconut, date palm, olive, sugarbeet, tea, and coffee; wood- or pulp-producing trees; vegetable crop plants such as legumes (for example, beans, peas, lentils, alfalfa, peanut), lettuce, asparagus, artichoke, celery, carrot, radish, the brassicas (for example, cabbages, kales, mustards, and other leafy brassicas, broccoli, cauliflower, Brussels sprouts, turnip, kohlrabi), edible cucurbits (for example, cucumbers, melons, summer squashes, winter squashes), edible alliums (for example, onions, garlic, leeks, shallots, chives), edible members of the Solanaceae (for example, tomatoes, eggplants, potatoes, peppers, groundcherries), and edible members of the Chenopodiaceae (for example, beet, chard, spinach, quinoa, amaranth); fruit crop plants such as apple, pear, citrus fruits (for example, orange, lime, lemon, grapefruit, and others), stone fruits (for example, apricot, peach, plum, nectarine), banana, pineapple, grape, kiwifruit, papaya, avocado, and berries; and ornamental plants including ornamental flowering plants, ornamental trees and shrubs, ornamental groundcovers, and ornamental grasses. Preferred dicot plants include, but are not limited to, canola, cotton, potato, quinoa, amaranth, buckwheat, safflower, soybean, sugarbeet, and sunflower, more preferably soybean, canola, and cotton. Preferred monocots include, but are not limited to, wheat, oat, barley, maize, rye, triticale, rice, ornamental and forage grasses, sorghum, millet, and sugarcane, more preferably maize, wheat, and rice.

Preferred but non-limiting examples of the plant of the invention are a tobacco plant (Nicotiana tabacum) or a potato plant (Solanum tuberosum).

In principle, the plant pest or plant pathogen may be a pest or pathogen of any of the plants mentioned herein, in particular of the preferred and/or specifically mentioned plants.

A target gene of interest may include any coding (preferred) or non-coding sequence from any species (including, but not limited to, eukaryotes such as fungi; plants, including monocots and dicots, such as crop plants, ornamental plants, and non-domesticated or wild plants; invertebrates such as arthropods, annelids, nematodes, and molluscs; and vertebrates such as amphibians, fish, birds, and mammals. Non-limiting examples of a non-coding sequence to be expressed by a gene expression element include, but not limited to, 5′ untranslated regions, promoters, enhancers, or other non-coding transcriptional regions, 3′ untranslated regions, terminators, introns, microRNAs, microRNA precursor DNA sequences, small interfering RNAs, RNA components of ribosomes or ribozymes, small nucleolar RNAs, and other non-coding RNAs. Non-limiting examples of a gene of interest further include, but are not limited to, translatable (coding) sequence, such as genes encoding transcription factors and genes encoding enzymes involved in the biosynthesis or catabolism of molecules of interest (such as amino acids, fatty acids and other lipids, sugars and other carbohydrates, biological polymers, and secondary metabolites including alkaloids, terpenoids, polyketides, non-ribosomal peptides, and secondary metabolites of mixed biosynthetic origin). A gene of interest can be a gene native to the plant in which the recombinant DNA construct of the invention is to be transcribed, or can be a non-native gene. A gene of interest can be a marker gene, for example, a selectable marker gene encoding antibiotic, antifungal, or herbicide resistance (e.g., glyphosate or dicamba resistance), or a marker gene encoding an easily detectable trait (e.g., phytoene synthase or other genes imparting a particular pigment to the plant), or a gene encoding a detectable molecule, such as a fluorescent protein, luciferase, or a unique polypeptide or nucleic acid “tag” detectable by protein or nucleic acid detection methods, respectively). Selectable markers are genes of interest of particular utility in identifying successful processing of constructs of the invention.

In many preferred embodiments, the target gene is an essential gene of the plant pest or plant pathogen. Essential genes include genes that are required for development of the pest or pathogen to a fertile reproductive adult. Essential genes include genes that, when silenced or suppressed, result in the death of the organism (as an adult or at any developmental stage, including gametes) or in the organism's inability to successfully reproduce (e.g., sterility in a male or female parent or lethality to the zygote, embryo, or larva). A description of nematode essential genes is found, e.g., in Kemphues K. “Essential Genes” (Dec. 24, 2005), WormBook, ed. The C. elegans Research Community, WormBook, doi/10.1895/wormbook.l.57.1, available on line at www.wormbook.org. Non-limiting examples of nematode essential genes include major sperm protein, RNA polymerase II, and chitin synthase (see, e.g., U.S. Patent Application Publication US 20040098761 A1); additional soybean cyst nematode essential genes are provided in U.S. patent application Ser. No. 11/360,355, filed 23 Feb. 2006, incorporated by reference herein. A description of insect genes is publicly available at the Drosophila genome database (available on line at flybase.bio.indiana.edu/). The majority of predicted Drosophila genes have been analyzed for function by a cell culture-based RNA interference screen, resulting in 438 essential genes being identified; see Boutros et al. (2004) Science, 303:832-835, and supporting material available on line at www.sciencemag.org/cgi/content/full/303/5659/832/DCI. A description of fungal essential genes is provided in the Database of Essential Genes (“DEG”, available on line at tubic.tju.edu.cn/deg/); see Zhang et al. (2004) Nucleic Acids Res., 32:D271-D272.

Target genes from pests can include invertebrate genes for major sperm protein, alpha tubulin, beta tubulin, vacuolar ATPase, glyceraldehyde-3-phosphate dehydrogenase, PvNA polymerase π, chitin synthase, cytochromes, miRNAs, miRNA precursor molecules, miRNA promoters, as well as other genes such as those disclosed in United States Patent Application Publication 2006/0021087 A1, PCT Patent Application PCT/US05/11816, and in Table II of United States Patent Application Publication 2004/0098761 A1, which are incorporated by reference herein. Target genes from pathogens can include genes for miRNAs, miRNA precursor molecules, fungal tubulin, fungal vacuolar ATPase, fungal chitin synthase, fungal MAP kinases, fungal Pacl Tyr/Thr phosphatase, enzymes involved in nutrient transport (e.g., amino acid transporters or sugar transporters), enzymes involved in fungal cell wall biosynthesis, cutinases, melanin biosynthetic enzymes, polygalacturonases, pectinases, pectin lyases, cellulases, proteases, genes that interact with plant avirulence genes, and other genes involved in invasion and replication of the pathogen in the infected plant.

Preferred but not-limiting examples of the target gene in accordance with the invention are ACT or SHR, in particular if the plant pest is an insect (like CPB, EPIC2B and PnPMA1), in particular if the plant pathogen is a fungus (like Phytophthora infestans).

In one aspect, the invention relates to a plastid as described and defined herein elsewhere; i.e. to a plastid comprising a dsRNA capable of silencing at least one target gene of a pest of a plant or of an agent causing a disease of a plant wherein said dsRNA comprises two (separate or covalently bound) complementary single-stranded RNA strands. What has been said herein elsewhere with respect to the plastid, dsRNA, target gene, plant pest/pathogen, the plant, etc. also applies here, mutatis mutandis.

For example, the plastid may be genetically engineered so that it produces/expresses the dsRNA. The dsRNA may be transcribed from the plastid's genome, for example from an encoding nucleotide sequence introduced therein (e.g. recombinant DNA construct). The plastid may comprise a nucleotide sequence which encodes and expresses the dsRNA.

In another aspect, the invention relates to a plant cell comprising the plastid of the invention. Again, what has been said herein elsewhere with respect to the plastid, target gene, plant pest/pathogen, plant, etc. also applies here, mutatis mutandis.

This invention also provides a transgenic plant cell having in its genome, in particular in the genome of its plastid(s), a recombinant DNA construct for plant cell transformation, including transcribable DNA including DNA that transcribes to an RNA for silencing a target gene of a pest or pathogen of a plant, wherein the RNA includes the dsRNA.

The transgenic plant cell can be an isolated plant cell (e.g., individual plant cells or cells grown in or on an artificial culture medium), or can be a plant cell in undifferentiated tissue (e.g., callus or any aggregation of plant cells). The transgenic plant cell can be a plant cell in at least one differentiated tissue selected from the group consisting of leaf (e.g., petiole and blade), root, stem (e.g., tuber, rhizome, stolon, bulb, and corm) stalk (e.g., xylem, phloem), wood, seed, fruit (e.g., nut, grain, fleshy fruits), and flower (e.g., stamen, filament, anther, pollen, carpel, pistil, ovary, ovules). Further provided is a transgenic plant containing the transgenic plant cell of this invention, that is, a transgenic plant having in its genome, in particular in the genome of its plastid(s), a recombinant DNA construct for plant cell transformation, including transcribable DNA including DNA that transcribes to an RNA for silencing a target gene of a pest or pathogen of a plant, wherein the RNA includes the dsRNA. The transgenic plant of the invention includes plants of any developmental stage, and includes a regenerated plant prepared from the transgenic plant cells claimed herein, or a progeny plant (which can be an inbred or hybrid progeny plant) of the regenerated plant, or seed of such a transgenic plant. Also provided and claimed is a transgenic seed having in its genome a recombinant DNA construct including transcribable DNA including DNA that transcribes to an RNA for silencing a target gene of a pest or pathogen of a plant, wherein the RNA includes the dsRNA and a transgenic plant grown from such transgenic seed.

In another aspect, the invention relates to a method of producing a plant, plastid or cell of the invention.

The method of producing a plant of the invention may comprise the steps of
(i) genetically engineering a plant cell so as to comprise a plastid comprising a dsRNA as described and defined herein; and
(ii) (re)generating from said plant cell a plant.

The method of producing a plant cell of the invention may comprise the steps of
(i) genetically engineering a plant cell so as to comprise a plastid comprising a dsRNA as described and defined herein (a plastid of the invention); and
(ii) (re)generating said plant cell.

The method of producing a plastid of the invention may comprise the step of genetically engineering a plastid so as to comprise a dsRNA as described and defined herein.

In the context of the invention, genetically engineering a plant, plant cell and/or plastid so as to comprise a plastid comprising a dsRNA as described and defined herein (a plastid of the invention) may be achieved by introducing into a plant or plant cell and/or, preferably, into a plastid (for example as comprised in the plant or plant cell) a nucleotide sequence (e.g. a recombinant DNA construct) encoding the dsRNA to be employed in accordance with the invention. The dsRNA may then be transcribed/expressed from said nucleotide sequence, preferably within said plastid, more preferably from the plastid's genome into which said nucleotide sequence (e.g. a recombinant DNA construct) has been integrated. Illustrative but non-limiting examples of such nucleotide sequences are the “ptDP”, “ptSL” and “ptHP” constructs described herein (cf. FIGS. 1A and 4).

In another aspect, the invention relates to a method of controlling a plant pest or a plant disease-causing agent (plant pathogen) as defined herein elsewhere comprising (the steps of)
(i) growing and/or providing a plant of the invention; and
(ii) allowing said pest or agent to affect said plant.

In another aspect, the invention relates to a method of protecting a plant from a plant pest or from a plant disease-causing agent (plant pathogen) as defined herein elsewhere comprising (the steps of)
(i) growing and/or providing a plant of the invention; and
(ii) allowing said pest or agent to affect said plant.

In another aspect, the invention relates to the use of a dsRNA as defined herein elsewhere for controlling a pest of a plant or a plant disease-causing agent affecting a plant, wherein said dsRNA is located in the plastids of said plant.

It is particularly envisaged in a preferred embodiment that the method of protecting of the invention is a method of complete or nearly complete protecting and that the controlling a plant pest or a plant disease-causing agent comes along with complete or nearly complete protection from the plant pest or plant disease-causing agent, respectively. “Complete protection” in this respect means that no (substantial) damage is caused to the plant by the plant pest/pathogen.

Again, what has been said herein elsewhere with respect to the plastid, dsRNA, target gene, plant pest/pathogen, plant, etc. also applies with respect to the other aspects of the invention described above, mutatis mutandis.

The present invention further relates to the following items:
1. A plant comprising a plastid comprising a double-stranded RNA (dsRNA) capable of silencing at least one target gene of a pest of a plant (plant pest) or of an agent causing a disease of a plant (plant pathogen), wherein said dsRNA comprises two complementary single-stranded RNA strands.
2. The plant of item 1, wherein said dsRNA comprises two separate complementary single-stranded RNA strands.
3. The plant of item 1 or 2, wherein said plastid is a chloroplast.
4. The plant of any one of items 1 to 3 which is a vascular plant.
5. The plant of any one of items 1 to 4, wherein said dsRNA is at least 50 basepairs in length.
6. The plant of any one of items 1 to 5, wherein said dsRNA is about 150-650 basepairs in length.
7. The plant of any one of items 1 to 6, wherein the sense strand of said dsRNA is at least 60% identical to an RNA transcribed from a nucleotide sequence of at least 50 contiguous nucleotides of said target gene.
8. The plant of any one of items 1 to 7, wherein at least one of said separate RNA strands comprises at least one stabilizing feature.
9. The plant of item 8, wherein said stabilizing feature is a stemloop structure.
10. The plant of any one of items 1 to 9, wherein each of said separate RNA strands comprises at least one stabilizing feature as defined in claim 8 or 9 at its 5′- and/or 3′-end.
11. The plant of any one of items 1 to 10, wherein said plastid is genetically engineered so as to comprise a nucleotide sequence encoding said dsRNA, wherein said dsRNA is transcribed from said nucleotide sequence.
12. The plant of any one of items 1 to 11, wherein said dsRNA is expressed by transcription from a nucleotide sequence flanked by two convergent promotors.
13. The plant of any one of items 1 to 12, wherein said plant pest is selected from the group consisting of:
   (i) an arthropod;
   (ii) a nematode; and
   (iii) a snail or slug.
14. The plant of item 13, wherein said arthropod is an insect or a mite.
15. The plant of item 14, wherein said insect is a Colorado potato beetle (Leptinotarsa decemlineata), including any juvenile stage of said beetle.
16. The plant of any one of items 1 to 12, wherein said plant pathogen is a fungal plant pathogen.
17. The plant of item 16, wherein said fungal plant pathogen is Phytophthora infestans.
18. The plant of any one of items 1 to 17, which is a potato plant or a tobacco plant.
19. The plant of any one of items 1 to 18, wherein said target gene is ACT, SHR, EPIC2B or PnPMA1.
20. A plastid as defined in any one of items 1 to 19.
21. A plant cell comprising a plastid of item 20.
22. A method of producing a plant of any one of items 1 to 19, comprising the steps of
   (i) genetically engineering a plant cell so as to comprise a plastid comprising a dsRNA as defined in any one of items 1, 2, 5 to 17 and 19; and
   (ii) (re)generating from said plant cell a plant.
23. A method of controlling a plant pest or a plant pathogen as defined in any one items 1 and 13 to 17 and/or protecting a plant from said plant pest or plant pathogen comprising the steps of
   (i) growing a plant of any one of items 1 to 19; and
   (ii) allowing said plant pest or plant pathogen to affect said plant.
24. Use of a dsRNA as defined in any one of items 1, 2, 5 to 17 and 19 for controlling a plant pest or a plant pathogen and/or for protecting a plant from said plant pest or plant pathogen,
   wherein said dsRNA is located in the plastids of said plant.

The present invention is further described by reference to the following non-limiting figures and examples.

The Figures show:

FIG. 1: Expression of dsRNAs in plastids. (A) Map of transformation vectors for dsRNA expression from the plastid genome. The cassettes designed to produce the three different types of dsRNAs (ptDP, ptSL and ptHP) are schematically depicted below the map, along with the expected structures and sizes of the dsRNAs. The location of the hybridization probe is shown as a black bar. The selectable marker gene aadA is driven by the psbA promoter (PpsbA) and fused to the 3′UTR of the rbcL gene (TrbcL) from Chlamydomonas reinhardtii. DNA sequences selected from CPB target genes (ACT, SHR and ACT+SHR fusion gene) are shown in orange. SL1, SL2: stemloop-encoding sequences; Prrn: tobacco rRNA operon promoter; TrrnB: rrnB terminator from E. coli; intron: first intron from the potato GA20 oxidase gene. (B) Example of a Southern blot to confirm transformation of the tobacco plastid genome, integration of the transgenes and homoplasmy. DNA was digested with BgIII and hybridized to a radiolabeled probe detecting the region of the plastid genome that flanks the transgene insert site. The absence of a hybridization signal for the wild-type genome indicates homoplasmy of all transplastomic lines. Note that the ptHP construct contains an internal BgIII site (FIG. 4C) and, therefore, the transplastomic Nt-ptHP lines produce a smaller restriction fragment than the Nt-ptDP and Nt-ptSL lines. (C) Northern blot analysis of dsRNA accumulation in transplastomic tobacco and potato lines. 5 μg total RNA were loaded in each lane, the band sizes of the RNA marker are given on the left. The ethidium bromide-stained gel prior to blotting is shown below each blot. The asterisk indicates a shorter-than-expected transcript species present in Nt-ptHP-ACT+SHR lines. Accumulation of some larger RNA species is likely due to read-through transcription, which is common in plastids (20, 21). Note that transplastomic lines independently generated with the same construct show identical transgene expression levels, due to targeting by homologous recombination and absence of epigenetic gene silencing mechanisms from plastids. (D) Quantification of dsRNA accumulation levels in transplastomic potato lines. 5 μg of total cellular RNA were loaded from the transplastomic lines. For semi-quantitative analysis, a dilution series of in vitro synthesized ssRNA was loaded. (E) Comparison of dsRNA accumulation levels in leaves and tubers of transplastomic potato lines. From each transformed line, leaves and tubers were harvested for total RNA isolation, and 5 μg of total cellular RNA were loaded per lane. The ethidium bromide-stained gel prior to blotting is shown below each blot.

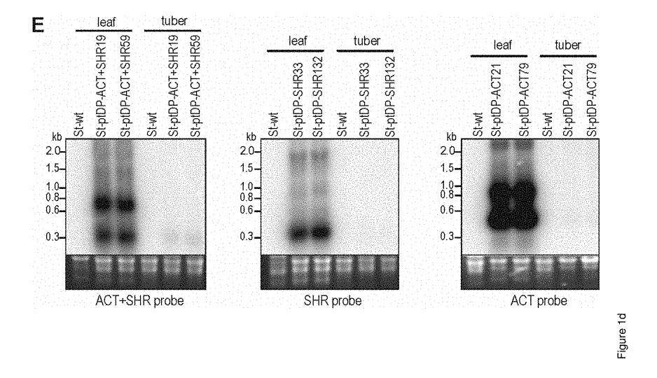

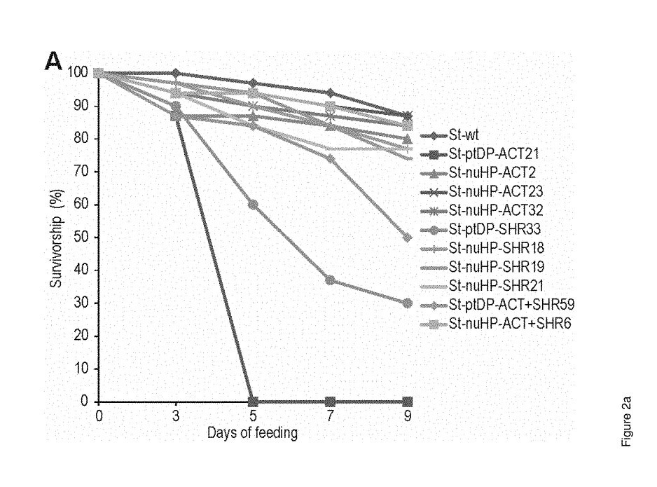

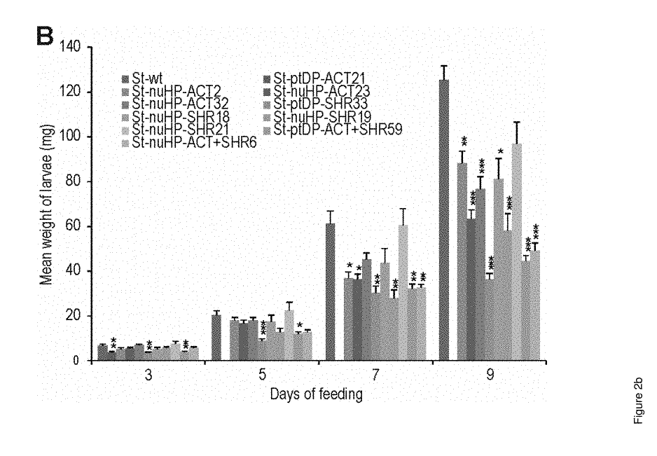

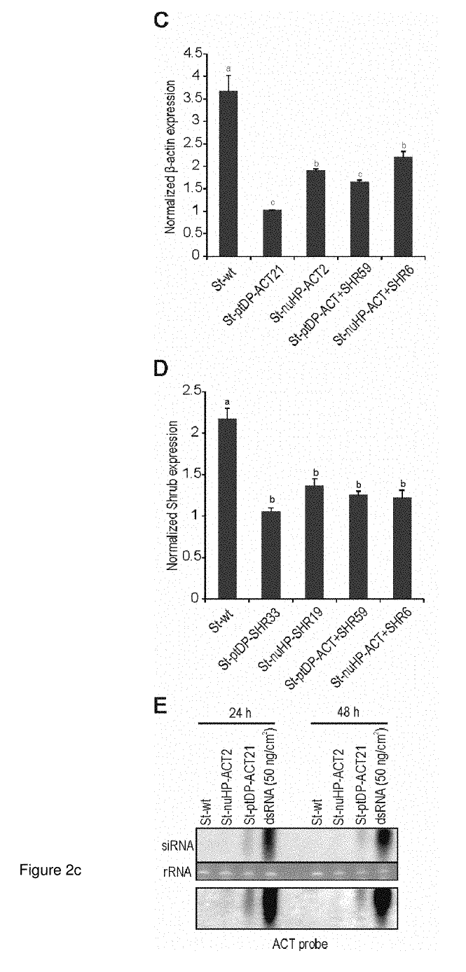

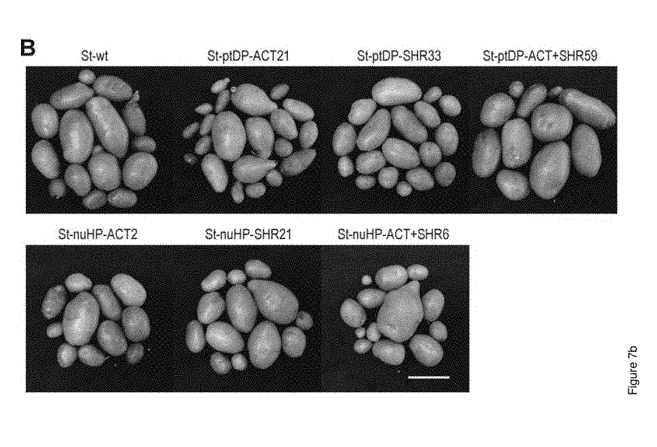

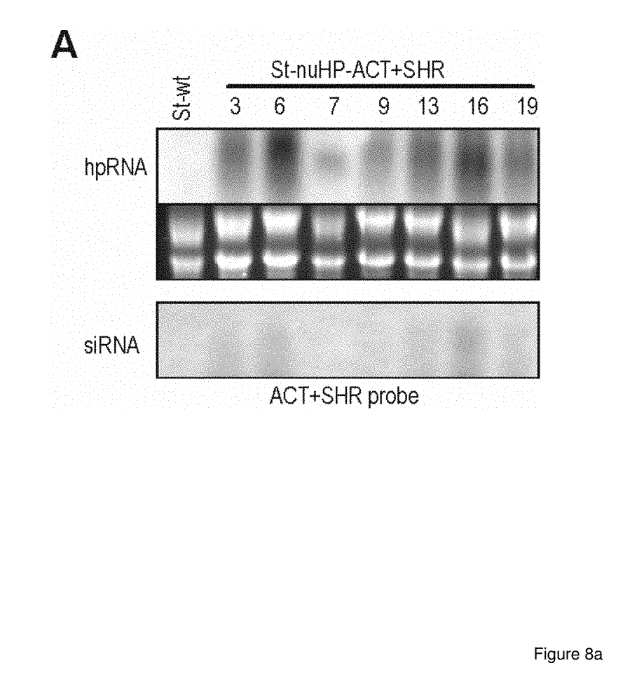

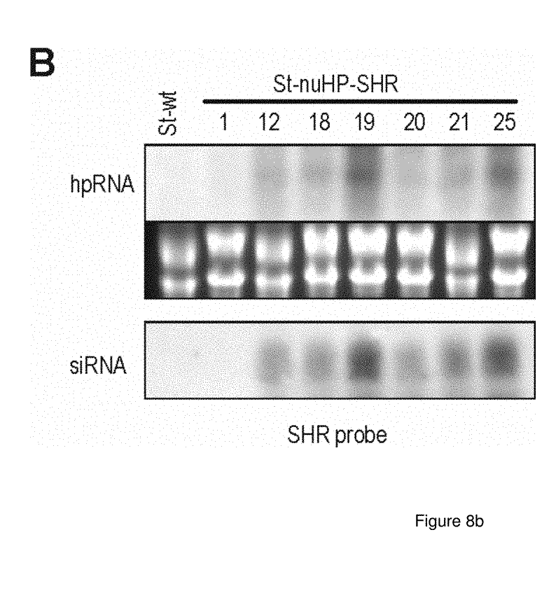

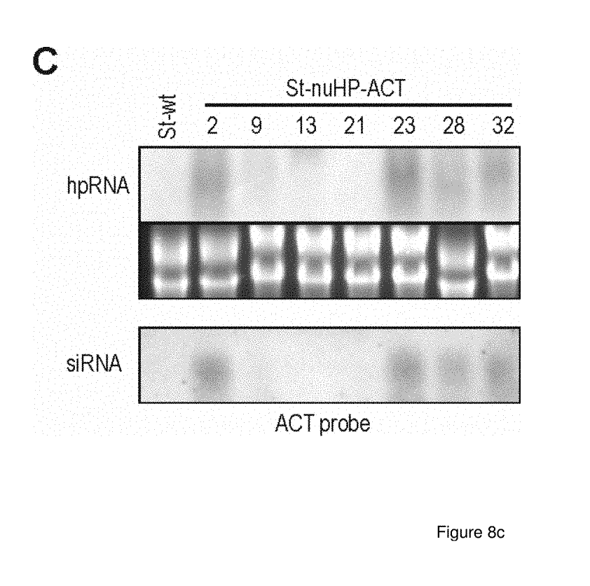

FIG. 2: Feeding assays of CPB larvae on transgenic and transplastomic potato plants. (A) Survivorship of first instar larvae upon feeding on detached leaves of wild-type, transplastomic and transgenic potato plants. (B) Growth of surviving larvae. The weight of survivors was determined after 3, 5, 7 and 9 days of feeding. Data are mean±SEM (n=30). Significant differences to the wild-type control were identified by ANOVA tests. * indicates a significant difference at P<0.05, ** indicates a significant difference at P<0.01, and *** indicates a significant difference at P<0.001. The best-performing nuclear transgenic lines were included in the assay (cf. FIGS. 8-10). Note that the weight of survivors in the assays with the transplastomic plants expressing ACT dsRNA (St-ptDP-ACT21) could only be measured till day 3, because all larvae were already dead at day 5 (cf. panel A). (C) Suppression of the β-actin gene in the gut of CPB larvae fed on transplastomic and transgenic potato plants. Relative expression values determined by qRT-PCR assays and normalized to two housekeeping genes are shown (for details, see Materials and Methods). Note that expression was measured at day 3 when most larvae fed on the transplastomic St-ptDP-ACT plants were still alive. Data represent mean and standard error from three biological replicates. The letters above each bar indicate the significance of the differences as determined by one way ANOVA in SPSS (P<0.05). (D) Suppression of the Shrub gene in the gut of CPB larvae fed on transplastomic and transgenic potato plants. Expression was determined at day 3 when most larvae fed on the transplastomic plants were still alive. (E) Induction of ACT mRNA degradation in the larval gut. RNA was extracted from gut tissue 24 h and 48 h after feeding of CPB larvae on wild-type, transplastomic or nuclear transgenic potato leaves. As an additional control, high concentrations of in vitro synthesized ACT dsRNA (50 ng/cm²) were painted onto wild-type leaves. siRNAs derived from the ACT mRNA were detected by northern blotting. As a loading control, the ethidium bromide-stained PAA gel prior to blotting (with an rRNA band of the cytosolic 80S ribosomes) is shown between a normal exposure of the blot (upper panel) and a strong exposure (lower panel). Note detection of ACT-derived siRNAs in gut tissue from larvae fed with transplastomic leaves, whereas siRNAs are below the limit of reliable detection in larvae fed with nuclear-transgenic leaves.

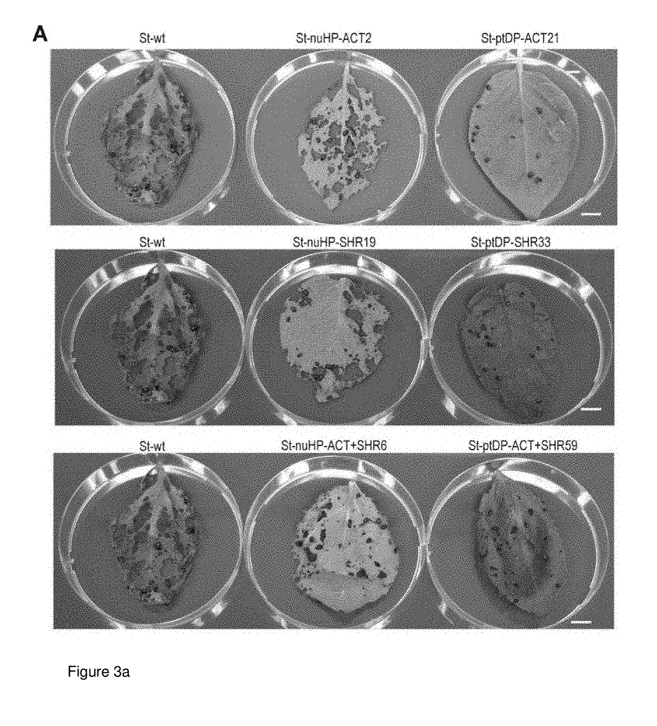

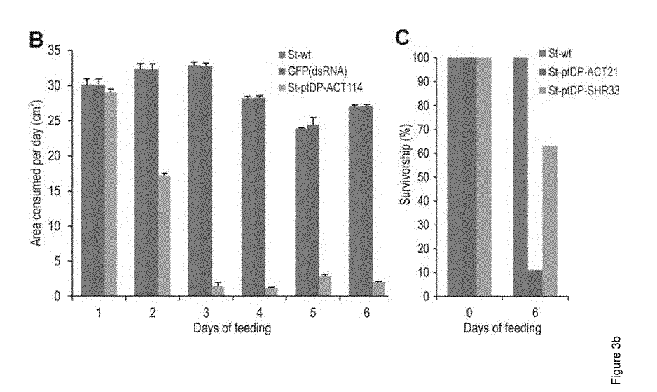

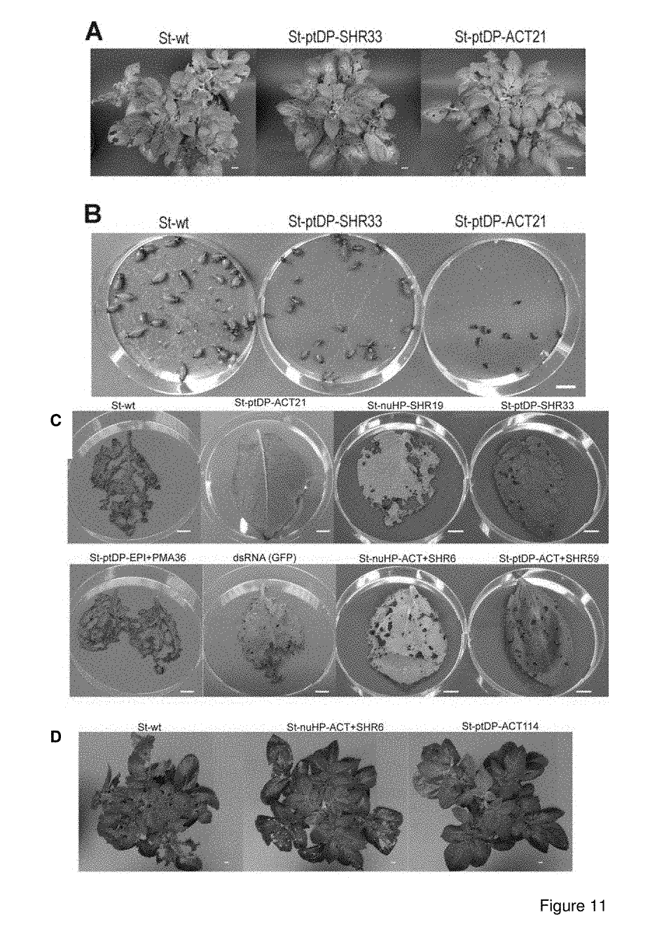

FIG. 3: Consumption of detached leaves of potato plants by CPB larvae and adult beetles, and survivorship of larvae upon feeding on whole plants. (A) Bioassay with detached leaves of wild-type, transgenic and transplastomic potato plants. Leaves were exposed to first instar CPB larvae, the photograph was taken at day 3. Note that almost no visible damage is seen in St-ptDP-ACT leaves. Scale bars: 1 cm. (B) Leaf area consumed by freshly emerged adult beetles fed on leaves of wild-type potato plants and transplastomic plants expressing ACT dsRNA (St-ptDP-ACT114). As an additional control, leaves painted with in vitro synthesized GFP-derived dsRNA were included. Data are mean±SD (n=12). (C) Survivorship of second instar CPB larvae after feeding on whole plants at day 6 (cf. FIG. 11).

FIG. 4: Transformation vectors for chloroplast and nuclear expression of dsRNAs and analysis of transplastomic potato lines by Southern blotting. (A) Physical maps of the targeting regions in the plastid genomes (ptDNA) of potato (St) and tobacco (Nt). Genes above the line are transcribed from left to right, genes below the line are transcribed in the opposite direction. BgIII restriction sites used for RFLP analysis of transplastomic lines are indicated and the sizes of the restriction fragments detected in Southern blot analyses are given. The location of the hybridization probe is also shown (black bar). (B) Map of the transformed region of the potato plastid genome. The sizes of the BgIII restriction fragments are given for all three transgenes (ACT, SHR and ACT+SHR fusion) expressed from the ptDP cassette. The selectable marker gene aadA is driven by the psbA promoter (PpsbA) and the 3′UTR of the rbcL gene (TrbcL) from Chlamydomonas. (C) Map of the transformed region of the tobacco plastid genome in Nt-ptDP, Nt-ptSL and Nt-ptHP transplastomic lines. The CPB transgenes are shown in orange, their orientation is indicated by arrows. SL1, SL2: stemloop-encoding sequences; Prrn: tobacco rRNA operon promoter; TrrnB: rrnB terminator from E. coli (dark blue); intron: first intron from the potato GA20 oxidase gene (light blue). (D) Map of the T-DNA locus in nuclear transgenic potato lines transformed with hairpin constructs (nuHP) for expression of ACT, SHR and the ACT+SHR fusion. CaMV 35S: 35S promoter from cauliflower mosaic virus (CaMV); T_{CaMV}: CaMV 35S terminator; 2×CaMV 35S: double 35S promoter from CaMV; Tocs: octopine synthase gene terminator from Agrobacterium tumefaciens; hpt: hygromycin resistance gene. (E) Example of a Southern blot to confirm transformation of the plastid genome in potato, integration of the transgenes by homologous recombination and homoplasmy. Total cellular DNA was digested with BgIII and hybridized to a radiolabeled probe detecting the region of the plastid genome that flanks the transgene insert site (cf. panels A-C). The absence of the 3 kb hybridization signal for the wild-type genome indicates homoplasmy of all transplastomic lines.

FIG. 5: In vitro dsRNA feeding assay. CPB second instar larvae were fed on young leaves of wild-type potato plants that had been painted with defined amounts of dsRNAs produced by in vitro transcription. The weight of the larvae was measured at the indicated time points. All data are means±SEM (n=30). The letters above each bar indicate the significance of the differences as determined by one way ANOVA in SPSS (Tukey's HSD test).

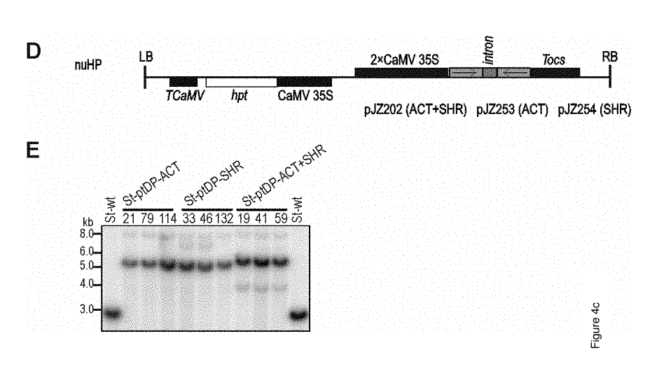

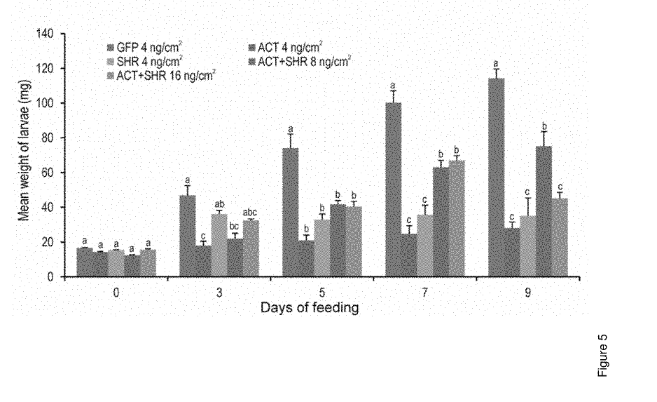

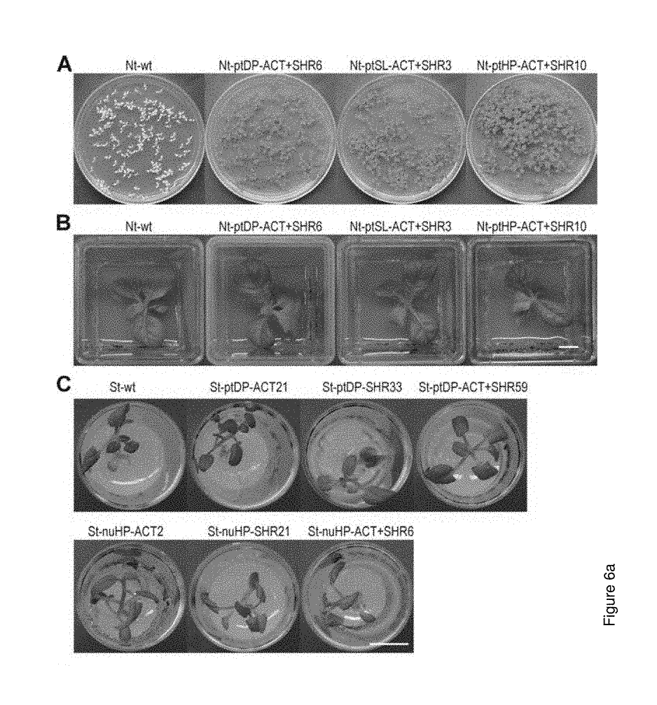

FIG. 6: Stable inheritance of plastid transgenes and wild-type-like phenotypes of transplastomic tobacco and potato lines. (A) Seed assays to confirm homoplasmy of transplastomic tobacco plants. Seeds obtained from wild-type plants (Nt-wt) and transplastomic plants expressing the three different types of dsRNA constructs (Nt-ptDP, Nt-ptSL, Nt-ptHP; FIG. 1A) were germinated on synthetic medium containing spectinomycin. Resistance of seedlings to the antibiotic and lack of segregation confirm the homoplasmic state of the transplastomic lines. (B) Phenotypes of transplastomic tobacco lines grown on synthetic medium. (C) Phenotypes of transplastomic potato lines (upper row) and transgenic potato lines (lower row) grown on synthetic medium. Transplastomic and transgenic lines for all target genes (ACT, SHR, ACT+SHR fusion) and a wild-type plant (St-wt) are shown. (D) Phenotypes of soil-grown transplastomic tobacco lines. (E) Phenotypes of soil-grown transplastomic (upper row) and transgenic (bottom row) potato lines. Scale bars: 1 cm.

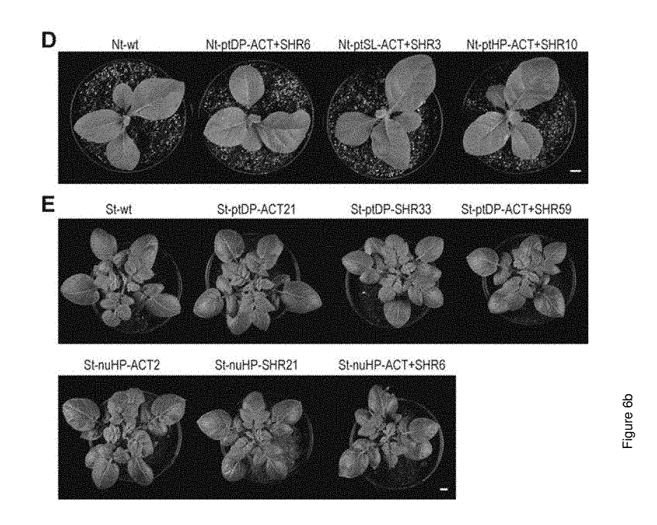

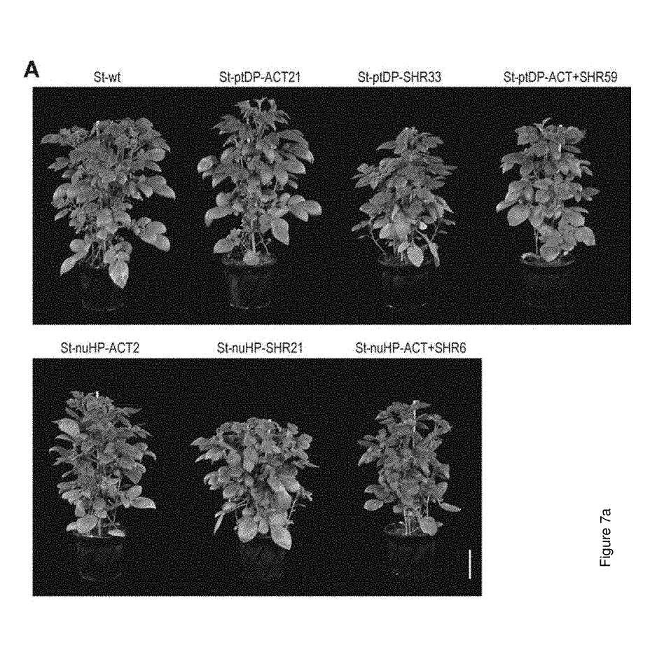

FIG. 7: Normal growth and tuber production of transgenic and transplastomic potato plants synthesizing dsRNAs against CPB target genes. (A) Phenotypes of transplastomic (upper row) and transgenic (lower row) potato plants after 9 weeks of growth under photoautotrophic conditions in soil. Scale bar: 10 cm. (B) Tubers harvested from wild-type, transplastomic (upper row) and transgenic (bottom row) potato plants. Scale bar: 5 cm.

FIG. 8: Northern blot analyses of hpRNAs and siRNAs in transgenic potato plants to identify highly expressing lines. (A) Accumulation of hpRNAs and siRNAs from the ACT+SHR transgene expressed in the nuclear genome. (B) Accumulation of hpRNAs and siRNAs from the SHR transgene. (C) Accumulation of hpRNAs and siRNAs from the ACT transgene. 20 μg of total cellular RNA were loaded in each lane of both the hpRNA and the siRNA blots. The ethidium bromide-stained agarose gels prior to blotting are shown below each hpRNA blot.

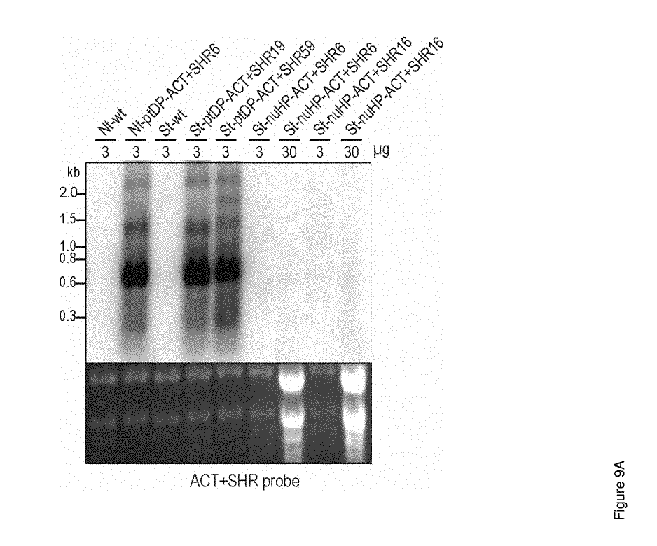

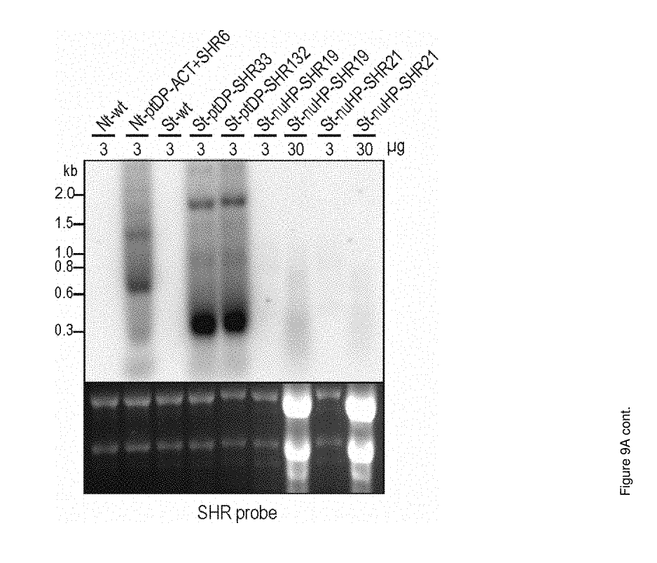

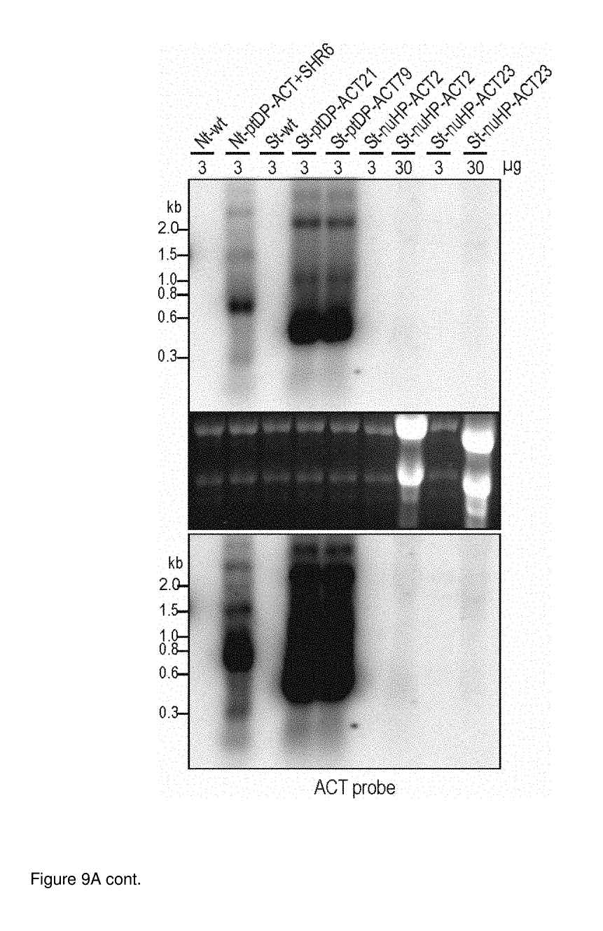

FIG. 9: Comparison of dsRNA accumulation in transplastomic and transgenic potato plants. (A) The amount of total RNA loaded in each lane is given (in μg). The ethidium bromide-stained gels prior to blotting are shown below each blot as a loading control. Note that ten times more RNA was loaded for the transgenic lines. The ACT blot was strongly overexposed (bottom panel) to detect at least some faint signals in the 30 μg samples of the nuclear transgenic lines. (B) Analysis of siRNA accumulation by northern blotting. Note that siRNAs accumulate only in the nuclear transgenic plants but not in the transplastomic plants, confirming that the dsRNAs produced in the plastid stay put. Thus, although the CPB ACT sequence used has some similarity to the potato ACT gene (66% over a stretch of 226 nt with the rest of the sequence having no significant similarity), it cannot even theoretically silence the plant's endogenous ACT gene, because the chloroplast-produced dsRNAs do not leak out into the cytosol.

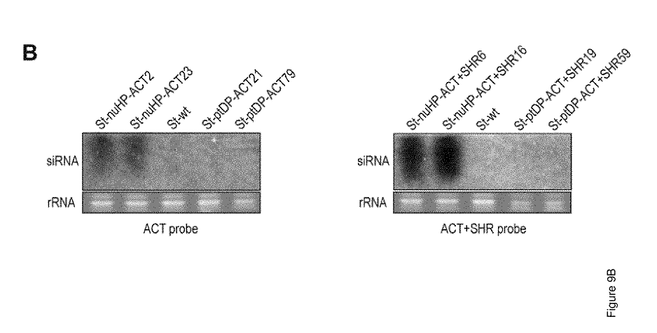

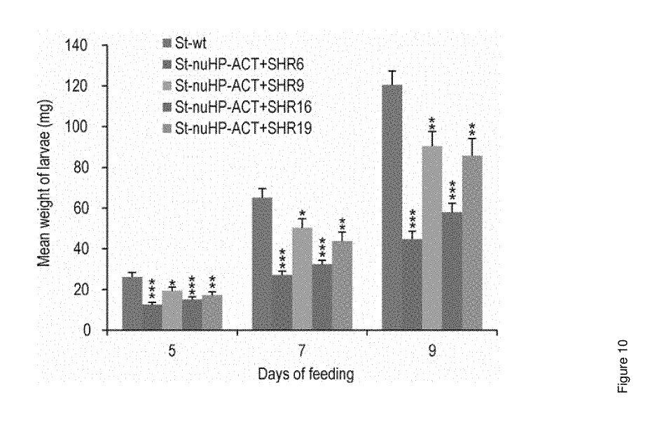

FIG. 10: Identification of the best-performing transgenic potato lines produced by nuclear transformation with the hairpin-type construct expressing the ACT+SHR fusion. Growth of first instar CPB larvae upon feeding on leaves of transgenic plants was recorded by measuring larval weight after 5, 7 and 9 days of feeding. Data represent mean±SEM (n=30). Significant differences between transgenic lines and wild-type control plants were verified by ANOVA SPSS (Tukey's HSD test). * indicates a significant difference at P<0.05, ** indicates a significant difference at P<0.01, and *** indicates a significant difference at P<0.001. Note that the growth retardation of the larvae correlates excellently with the hpRNA and siRNA accumulation levels in the different transgenic lines (cf. FIG. 8A).

FIG. 11: Exposure of whole potato plants to second instar CPB larvae—Bioassay with detached leaves and exposure of whole potato plants to second instar CPB larvae. (A) Damage to wild-type and transplastomic potato plants (St-ptDP-ACT21 and St-ptDP-SHR33). Second instar CPB larvae (n=35) were randomly release on the top leaves of the plants. The photograph was taken 6 days after larval release. (B) CPB larvae collected from the plants at day 6. Scale bars: 1 cm. (C) Examples of bioassays with detached leaves of wild-type potato plants and nuclear transgenic and transplastomic leaves expressing dsRNA. Leaves were exposed to first instar CPB larvae, replaced with fresh young leaves every day, and the photograph was taken at the end of day 3 (cf. FIG. 3A). Note that almost no visible damage is seen in St-ptDP-ACT leaves. As additional controls for specificity, wild-type leaves painted with dsRNA derived from the gfp gene and a transplastomic line expressing as dsRNA derived from Phytophthora infestans gene sequences (with no significant homology to CPB genes; St-ptDP-EPI+PMA) were included. For clarity, larvae were removed from leaves with no visible or massive damage prior to photographing. (D,A) Damage to wild-type, nuclear-transgenic (St-nuHP-ACT+SHR6) and transplastomic potato plants (St-ptDP-ACT114, St-ptDP-SHR33 and St-ptDP-ACT21). (D) Second instar CPB larvae (n=40) were randomly released on the top leaves of the plants. The photograph was taken 5 days after larval release. (A) Second instar larvae (n=35) were randomly released and the photograph was taken after 6 days. (B) CPB larvae collected from the plants shown in panel C at day 6. Scale bars: 1 cm.

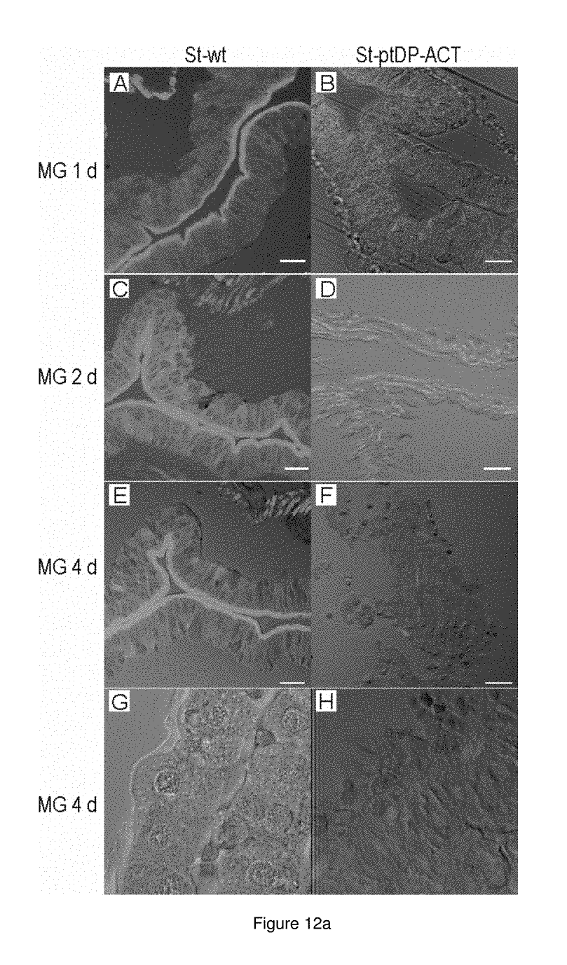

FIG. 12: Rapid disruption of β-actin filaments in different tissues of potato beetles after feeding on transplastomic potato plants. Midgut (MG; A-H), hindgut (HG; I-N) and Malpighian tubules (MT; O-P) of third instar CPB larvae were stained with phalloidin-FITC after 24 h (A-B), 48 h (C-D) and 96 h (E-P) of feeding on leaves of wild-type potato plants (St-wt) and transplastomic plants expressing ACT dsRNA (St-ptDP-ACT). Scale bars: 25 μm.

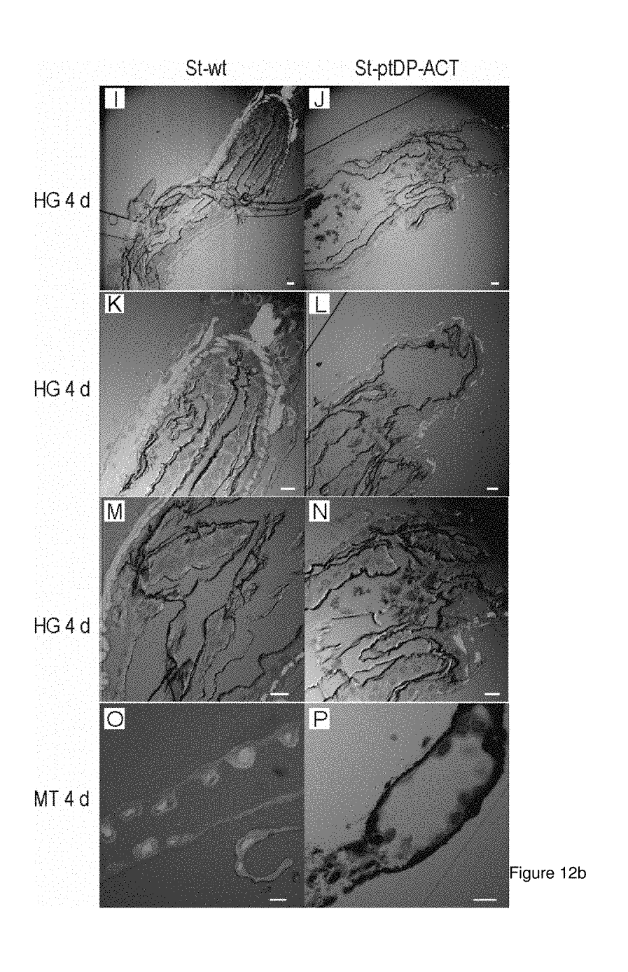

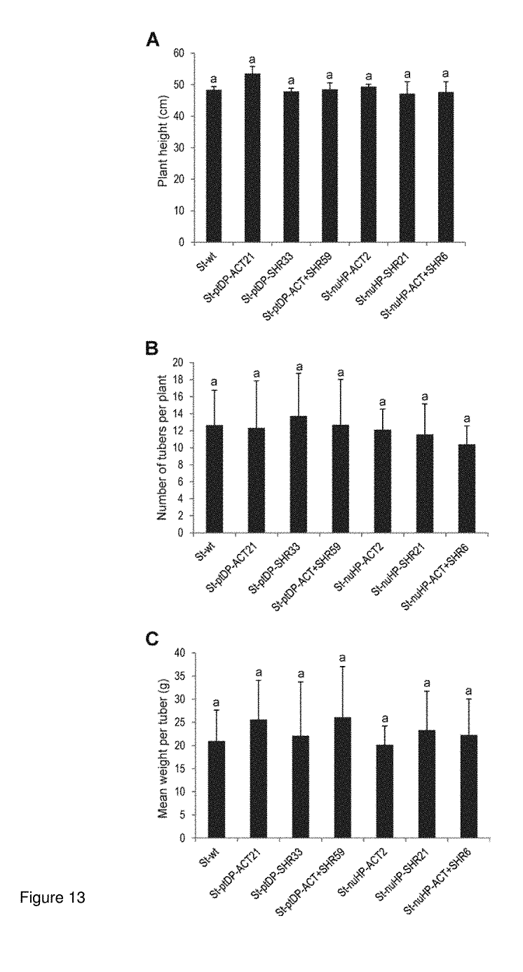

FIG. 13: Quantitative analysis of phenotypic traits in transplastomic and nuclear transgenic potato plants expressing dsRNAs targeted against CPB genes. Plants were grown in the greenhouse in standard pots (top diameter: 18 cm, bottom diameter: 14 cm; height: 16 cm) under a 16 h light/8 h dark regime at 18-20° C. and a relative humidity of 50-60%. St-wt: wild-type control plants. (A) Measurement of plant height at the onset of flowering. (B) Determination of the number of tubers produced per plant. (C) Measurement of the average tuber weight. The letter a indicates the absence of a significant difference (P>0.05; n=4-6). Data represent mean±SD.

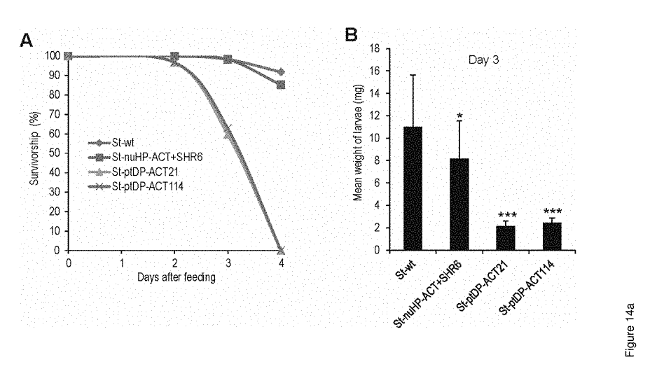

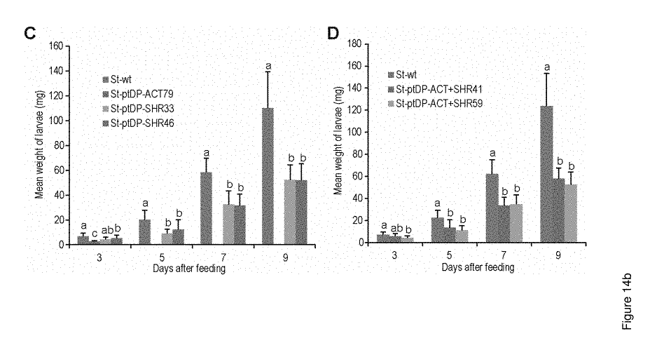

FIG. 14: Analysis of additional transplastomic potato lines in feeding assays with CPB larvae (cf. FIG. 2/3). (A) Survivorship of first instar larvae upon feeding on detached leaves of two independently generated transplastomic St-ptDP-ACT lines. For comparison, the wild type (St-wt) and a strong nuclear transgenic line were included. Note that the two transplastomic lines show no difference. This was expected because (i) transgene integration into the plastid genome occurs by homologous recombination, and (ii) plastid transgenes are not subject to expression variation resulting from position 51 effects and/or transgene silencing. (B) Mean weight of larvae after 3 days of feeding on St-ptDP-ACT lines. The best-performing nuclear line (St-nuHP-ACT+SHR6) was included for comparison. Significant differences to the wild-type control were identified by ANOVA tests. * indicates a significant difference at P<0.05, and *** indicates a significant difference at P<0.001. Note that later time points could not be investigated, because all larvae were already dead after 4-5 days (cf. FIG. 2/3). (C) Growth of surviving larvae upon feeding on two independently generated St-ptDP-SHR lines. The wild type (St-wt) and the St-ptDP-ACT79 line were included as controls. (D) Growth of surviving larvae upon feeding on two independently generated St-ptDP-ACT+SHR lines. The weight of survivors was determined after 3, 5, 7 and 9 days of feeding. Data are mean±SD (n=30). Significant differences to the wild-type control were identified by ANOVA tests (P<0.05).

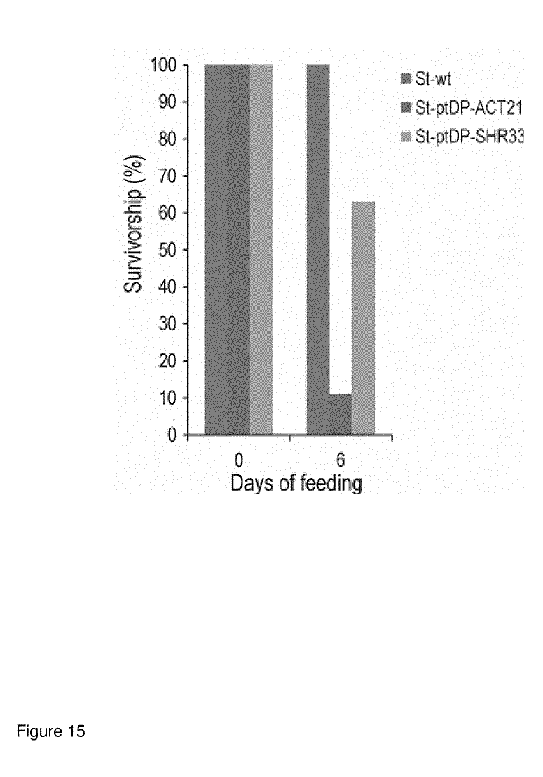

FIG. 15 Survivorship of second instar CPB larvae after feeding on whole plants at day 6 (cf. FIG. 11A). Wild-type potato plants, transplastomic plants expressing ACT dsRNA (St-ptDP-ACT21) and transplastomic plants expressing SHR dsRNA (St-ptDP-SHR33) were analyzed.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### EXAMPLE 1: MATERIALS AND METHODS

### Plant Material and Growth Conditions

To generate leaf material for biolistic plastid transformation experiments, tobacco plants (Nicotiana tabacum cv. Petit Havana) were grown under aseptic conditions on agar-solidified MS medium supplemented with 30 g/L sucrose (22). Potato (Solanum tuberosum cv. Desiree) plants for nuclear and chloroplast transformation experiments were grown on the same medium but at lower sucrose concentration (20 g/L). Transgenic and transplastomic lines were rooted and propagated on the same media in the presence of the appropriate antibiotic (spectinomycin or hygromycin). Rooted plantlets were grown in soil under standard greenhouse conditions. Inheritance patterns in transplastomic tobacco lines were analyzed by germination of surface-sterilized seeds on Petri dishes containing MS medium supplemented with spectinomycin (500 mg/L).

### Construction of Transformation Vectors

The plastid transformation vectors constructed in this study are based on a modified version of the previously described plasmid pKP9 (23). The aadA cassette in pKP9 was replaced by a modified cassette consisting of the Chlamydomonas reinhardtii PpsbA promoter, the coding region of the selectable marker gene aadA and the 3′UTR of the rbcL gene from Chlamydomonas reinhardtii (24, 25). The cassette was excised from a plasmid clone with the restriction enzymes SpeI and SmaI, followed by a fill-in reaction with the Klenow fragment of DNA polymerase I to generate blunt ends, and then cloned into a progenitor clone of pKP9 that was cut with the restriction enzyme Ec113611. A clone was selected which contained the aadA cassette in the opposite orientation of the upstream trnfM gene, yielding plastid transformation vector pJZ100 (FIG. 1A).

Target gene selection for RNA interference was based on previous reports (12, 3). A DNA fragment covering 297 bp of the β-actin gene (ACT) and 220 bp of the Shrub gene (SHR) from Leptinotarsa decemlineata was chemically synthesized as a fusion (ACT+SHR) with a 5′ extension (5′-GCATGCCTGCAG-3′; introducing SphI and PstI restriction sites for cloning purposes) and a 3′ extension (5′-AGATCT-3′; introducing a BgIII restriction site for cloning), and ligated into vector pUC57 (GenScript, Piscataway, N.J., USA), generating plasmid pJZ191. The ACT fragment covers nucleotides −49 to +248 of the 5′UTR and coding region of the β-actin cDNA, the SHR fragment covers nucleotides+179 to +398 of the coding region of the Shrub cDNA.

To assemble the ptDP constructs for dsRNA expression from convergent promoters, two copies of the plastid Prrn promoter were amplified. One copy was amplified with primer pair Prrn(HindIII)-F/Prrn(SphI)-R, introducing HindIII and SphI restriction sites with the primer sequences (Table 1). The PCR product was cloned as HindIII/SphI fragment into the similarly cut cloning vector pUC19, generating plasmid pJZ11. Subsequently, the ACT+SHR fragment was excised from pJZ191 as SphI/BgIII fragment and cloned into pJZ11 digested with SphI and BamHI, resulting in plasmid pJZ19. The second Prrn promoter copy was amplified using primer pair Prrn(EcoRI)-F/Prrn(SacI)-R (Table 1). The PCR product was digested with EcoRI and SacI, and cloned into the similarly cut plasmid pJZ19, producing plasmid pJZ193. The dsRNA expression cassette was then excised from pJZ193 as EcoRI/HindIII fragment and subcloned into pBluescript KS(−) digested with the same enzymes, resulting in plasmid pJZ197. Finally, the dsRNA cassette was excised from pJZ197 as NotI/XhoI fragment and inserted into the similarly cut plastid transformation vector pJZ100, producing vector pJZ199. ACT and SHR gene fragments were obtained by PCR amplification with primer pairs actin(SbfI)-F/actin(SacI)-R and shrub(SbfI)-f/shrub(SacI)-R, respectively (Table 1), using plasmid pJZ191 as template. The resulting PCR products were digested with SbfI and SacI, and cloned into the similarly cut vector pJZ199 to replace with ACT or SHR, generating plastid transformation vectors pJZ237 and pJZ238, respectively. To assemble the ptSL construct (designed to express dsRNAs with flanking stem-loop structures), one of the two Prrn promoter copies (including a sequence folding into a 24 bp stem-loop structure at the RNA level) was amplified using primers Prrn(HindIII)-F and PrrnSL1 (PstI)-R (Table 1). The resulting PCR product was digested with HindIII and PstI and ligated into the similarly cut cloning vector pUC19, generating plasmid pJZ10. Subsequently, the ACT+SHR fragment was excised from pJZ191 as PstI/BamHI fragment and cloned into the similarly cut pJZ10, producing plasmid pJZ14. The second Prrn promoter copy (also including a sequence folding into a 24 bp stem-loop structure at the RNA level) was amplified with primer pair Prrn(EcoRI)-F/PrrnSL2 (BamHI)-R (Table 1). The PCR product was digested with EcoRI and BamHI and ligated into pJZ14 cut with the same enzyme combination, resulting in plasmid pJZ192. The dsRNA-SL expression cassette was then excised from pJZ192 as EcoRI/HindIII fragment and subcloned into pBluescript KS(−), generating plasmid pJZ196. Finally, the dsRNA-SL cassette was excised from pJZ196 as NotI/XhoI fragment and inserted into plastid transformation vector pJZ100, generating vector pJZ200.

To assemble the ptHP construct for dsRNA expression as a hairpin RNA structure, the first intron from the potato gibberellin 20 (GA20) oxidase gene was excised from a plasmid clone (pUC-RNAi; 26) as PstI/BamHI fragment and inserted into the similarly cut vector pJZ11, generating plasmid pJZ158. The rrnB terminator (TrrnB) from Escherichia coli was amplified with primer pair TrrnB(SacI)-F/TrrnB(EcoRI)-R (Table 1), using plasmid pNtcC1-TrrnB (27) as template. The obtained PCR product was cloned as SacI/EcoRI fragment into pJZ158, producing plasmid pJZ171. The ACT+SHR sequence was excised from pJZ191 as SphI/BgIII fragment and cloned into the similarly cut pJZ171, generating pJZ194. A second copy of the ACT+SHR sequence was amplified with primer pair act+shr(SacI)-F/act+shr(SmaI)-R (Table 1). The PCR product was cloned (in antisense orientation) as SacI/SmaI fragment into the similarly cut pJZ194, generating plasmid pJZ216. The hpRNA expression cassette was subsequently excised from pJZ216 as EcoRI/HindIII fragment and subcloned into pBluescript KS(−), generating plasmid pJZ219. Finally, the hpRNA cassette was excised from pJZ219 as NotI/XhoI fragment and inserted into plastid transformation vector pJZ100, generating vector pJZ222.

For expression of hairpin-type dsRNAs in the nucleus (nuHP constructs), the ACT and SHR fragments were amplified with primer pairs actin(XbaI)-F/actin(BgIII)-R and shrub(XbaI)-F/shrub(BamHI)-R, respectively (Table 1). The ACT PCR product was cloned as XbaI/BgIII fragment into vector pUC-RNAi (26) cut with XbaI and BamHI, generating plasmid pJZ249. The SHR PCR product was cloned as XbaI/BamHI fragment into the similarly cut vector pUC-RNAi, producing plasmid pJZ250. The second ACT fragment was amplified with primers actin(XhoI)-F and actin(BgIII)-R (Table 1), and ligated as XhoI/BgIII fragment (in antisense orientation) into the similarly disgested vector pJZ249, generating plasmid pJZ251. The second SHR fragment was amplified with primer pair shrub(XhoI)-F/shrub(BamHI)-R (Table 1). The obtained PCR product was then cloned (in antisense orientation) as XhoI/BamHI fragment into vector pJZ250 that had been digested with XhoI and BgIII, generating plasmid pJZ252. Finally, the ACT and SHR sequences were excised as XhoI/XbaI fragments from pJZ251 and pJZ252, respectively, and cloned into vector pEZR(H)-LN (a kind gift from Dr. Staffan Persson, MPI-MP) cut with Sail and XbaI, generating nuclear transformation vectors pJZ253 and pJZ254. The ACT+SHR sequence was excised as PstI/SacI fragment from pJZ216, followed by blunting with Klenow enzyme and cloning into the SmaI/XbaI digested and blunted vector pEZR(H)-LN. A clone was selected in which the GA20 intron has the same orientation as the CaMV35S promoter, yielding nuclear transformation vector pJZ202.

### Construction of Plasmid Vectors for In Vitro Transcription

To construct vectors for in vitro synthesis of ssRNA, the ACT+SHR sequence was excised from pJZ191 as PstI/BamHI fragment and ligated into the similarly cut cloning vector pBluescript KS(−), resulting in plasmid pKS_ACT+SHR. The ACT sequence was amplified with primer pair actin(XhoI)-F/actin(BgIII)-R (Table 1). The PCR product was digested with XhoI and BgIII, and cloned into pBluescript KS(−) cut with XhoI and BamHI, generating plasmid pKS_ACT. The SHR sequence was amplified with primer pair shrub(XhoI)-F/shrub(BamHI)-R (Table 1). The PCR product was digested with XhoI and BamHI and cloned into the similarly cut pBluescript KS(−), generating plasmid pKS_SHR.

### Plastid and Nuclear Transformation

For tobacco plastid transformation, young leaves from plants grown under aseptic conditions were bombarded with plasmid DNA-coated gold particles using a PDS1000/He particle delivery system equipped with a Hepta adaptor (BioRad, Hercules, Calif., USA). Primary spectinomycin-resistant lines were selected on RMOP medium containing 500 mg/L spectinomycin (28). For each construct, several independent transplastomic lines were subjected to two additional rounds of regeneration on spectinomycin-containing medium to select for homoplasmy.

For potato plastid transformation, a published protocol (13) was slightly modified. The basic media (BM) for potato regeneration contained MS salts supplemented with B5 vitamins (pH adjusted to 5.7), and were solidified with 0.6% Micro agar (Duchefa). Medium StM1 consists of BM, 3% sucrose, 0.1 M sorbitol and 0.1 M mannitol. Medium StM2 contains BM, 3% sucrose, 2 mg/L 2,4-diclorophenoxyacetic acid (2,4 D), 0.8 mg/L zeatin riboside and 400 mg/L spectinomycin. Medium StM3 contains BM, 1.6% glucose, 2 mgl/L indole-3-acetic acid (IAA), 3 mg/L zeatin riboside, 1 mg/L gibberellic acid (GA3) and 400 mg/L spectinomycin. Medium StM4 contains BM, 3% sucrose, 0.1 mg/L IAA, 3 mg/L zeatin riboside and 400 mg/L spectinomycin. For transformation, young leaves from aseptically grown potato plants were incubated for 24 h on StM1 medium in the dark. After biolistic transformation, leaves were incubated for up to 1 day in the dark, then cut into pieces of 3×3 mm, transferred to StM2 medium and incubated under dim light (˜10 μmol photons m⁻² s⁻¹) in a 16 h light/8 h dark regime for 1 month. Subsequently, the leaf pieces were transferred to StM3 medium and subcultured every 4 weeks until resistant calli or shoots appeared. Resistant material was transferred to StM4 medium, and incubated for 1 to 3 months to induce shoot regeneration and multiplication. To stimulate rooting, regenerated shoots were transferred to MS medium with 3% sucrose and 400 mg/L spectinomycin. Finally, rooted plantlets were transferred to soil and grown to maturity. Homoplasmy was confirmed by Southern blotting.

Nuclear transgenic potato plants were generated by Agrobacterium-mediated transformation (29). Transgenic plants were identified by hygromycin selection and initially tested for the presence of the transgene by PCR assays. The transgenic status was further confirmed by RNA gel blot analyses.

### Isolation of Plant Nucleic Acids and Gel Blot Analysis

Total DNA from tobacco or potato plants was extracted from young leaves of soil-grown plants by a cetyltrimethylammonium bromide (CTAB)-based method (30). For DNA gel blot analysis, samples of 5 μg of total cellular DNA were digested with the restriction enzyme BgIII, separated by gel electrophoresis in 0.8% agarose gels and transferred onto Hybond nylon membranes (GE Healthcare, Buckinghamshire, UK) by capillary blotting. A 550 bp PCR product generated by amplification of a portion of the psaB coding region (31) was used as RFLP probe to verify plastid transformation and assess the homoplasmic status of transplastomic lines.

For RNA gel blot analysis, total cellular RNA was extracted using the peqGOLD TriFast reagent (Peqlab, Erlangen, Germany) from leaf samples of soil-grown tobacco or potato plants. Total RNA from potato tubers was isolated with the NucleoSpin RNA Plant kit (Macherey-Nagel, Duren, Germany) following the instructions of the supplier. RNA samples were separated by electrophoresis in 1% formaldehyde-containing agarose gels and blotted onto Hybond nylon membranes (GE Healthcare). For siRNA analysis, samples of 20 μg of total cellular RNA were separated in 14% polyacrylamide gels with 0.3 M sodium acetate and 7 M urea as gel buffer and 0.3 M sodium acetate (pH 5.0) as running buffer. The separated RNA samples were electroblotted onto Hybond nylon membranes in blotting buffer (10 mM Tris-acetate pH 7.8, 5 mM sodium acetate, 0.5 mM EDTA) at 40 V for 2 h at 4° C. (32) and subsequently cross-linked to the membrane by UV light.

PCR products generated by amplification with gene-specific primers were used as hybridization probes. [α³²P]dCTP-labeled probes were generated using the Multiprime DNA labeling system (GE Healthcare). Hybridizations were performed at 65° C. for standard Southern and northern blots and at 42° C. for siRNA blot analysis.

### In Vitro RNA Synthesis

For ssRNA synthesis by in vitro transcription from plasmids, 1 μg of plasmid DNA from clones pKS_ACT+SHR, pKS_ACT and pKS_SHR was linearized with XbaI. The linearized DNA fragments were purified using the NucleoSpinR Gel and PCR clean-up kit (Macherey-Nagel). In vitro transcription reactions were performed with T3 RNA polymerase (Thermo Scientific, Waltham, Mass., USA) following the manufacturer's instructions. The RNA yield was determined with a NanoDrop ND-1000 spectrophotometer.

In vitro synthesis of dsRNAs for insect feeding assays was carried out with the T7 RiboMAX™ express RNAi system (Promega, Mannheim, Germany) according to the manufacturer's protocol. pJZ191 plasmid DNA was used to amplify templates for in vitro transcription. The minimal T7 promoter sequence (5′-TAATACGACTCACTATAGG-3′) was added to the 5′ end of forward and reverse primers (Table 1).

### Insect Bioassays

A strain of Colorado potato beetle (Leptinotarsa decemlineata) was kindly provided by the Julius Kuhn Institute, Federal Research Centre for Cultivated Plants, Kleinmachnow, Germany. The insects were reared in the lab on wild-type potato plants (Solanum tuberosum L., cv. Delana or Desiree). CPB larvae were hatched from eggs, and neonates were reared on potato leaves at 26° C. under a 16 h light/8 h dark cycle.

To obtain standardized larvae for growth and survival assays on transplastomic and transgenic potato plants, CPBs were fed on wild-type potato plants and adults were allowed to lay eggs. The eggs were collected and transferred onto fresh wild-type potato leaves for hatching. First instar larvae were allowed to feed on young leaves of two-month old transplastomic or transgenic potato plants and wild type plants as a control. For each feeding experiment, synchronized groups of larvae were selected, weighed individually and divided into three groups (each group containing 10-20 individuals and serving as a biological replicate). After feeding on detached potato leaves for 3, 5, 7 and 9 days, larvae were weighed, and midgut and carcass tissues were taken from dissected larvae for further analysis. Similarly, adult CPBs were used to feed on transplastomic plants (St-ptDP-ACT). To calculate the consumed leaf area, the leaves were photographed before and after feeding by CPB and the consumed area was determined using the Sigma Scan Pro5 software. Statistical analysis was performed with one-way ANOVA (SPSS software) and results are presented as means±standard deviation.

For larval performance assays onto potato leaves painted with dsRNA, in vitro synthesized dsRNA was painted on young potato leaves in defined amounts per leaf area. To this end, fresh potato leaflets were arranged in a circle of about 23 cm² surface area and dsRNA (diluted in water) was painted onto the leaf surface to final concentrations of 4, 8 or 16 ng per cm². Second instar larvae were weighed after 0, 3, 5, 7 and 9 days of feeding on dsRNA-painted leaves. The larvae were divided into three groups (for three biological replicates) and each group had 10-20 larvae per treatment. dsRNA derived from the gfp coding region was used as a control. The leaves were replaced with fresh dsRNA-painted leaves every 24 hours.

RNA Extraction from CPB Larvae

Larvae were dissected in ice-cold Schneider fs insect medium (Sigma-Aldrich, St. Louis, Mo., USA). Larval gut, Malpighian tubules and the rest of the body were isolated as described previously (33, 34) and placed in 100 μl of ice-cold Schneider's medium in separate Eppendorf tubes. Immediately after collection, the tissues were flash-frozen in liquid nitrogen and stored at −80° C. until use. RNA was extracted from tissue samples with Trizol (Invitrogen, Carlsbad, Calif., USA) following the manufacturer's instructions. RNA integrity and quantity were checked on an Agilent 2100 Bioanalyzer using the RNA Nano chips (Agilent Technologies, Santa Clara, Calif., USA). RNA was then precisely quantified with a NanoDrop ND-1000 spectrophotometer.

Quantitative Real-Time PCR (qRT-PCR)

qRT-PCR was used to assess transcript levels of ACT and SHR in gut tissues. The software Primer-3 (http://frodo.wi.mit.edu/) was used to design the primers for qPCR analysis (for primer sequences, see Table 1). Reverse transcription reactions were performed with 500 ng of total RNA and oligo d(T) primer using the First Strand cDNA Synthesis kit (Fermentas) according to the manufacturer's protocol. qRT-PCR was done in optical 96-well plates on a MX3000P Real-Time PCR Detection System (Stratagene) using the ABsolute qPCR SYBR Green Mix (Thermo Scientific) to monitor double-stranded DNA synthesis in combination with ROX Passive Reference Dye. Amplification conditions were 10 min at 95° C., followed by 40 cycles at 95° C. for 30 s, 60° C. for 30 s and 72° C. for 30 s. Melt curve analysis was performed in order to assess the specificity of amplification. Results were normalized to the mRNA levels of the CPB genes encoding ribosomal protein S18 (RPS18) and ribosomal protein S4 (RPS4) as housekeeping genes (Table 1), and relative mRNA accumulation levels were calculated according to the delta-delta Ct method. Each experiment was repeated with three independently isolated mRNA samples (biological replicates), and each reaction was repeated 3 times to minimize intra-experiment variation (technical replicates). All results were analyzed with the qBase software.

### Histological Analysis of Actin Filaments in Larval Tissues

Third instar larvae fed on wild type and transplastomic potato plants (St-ptDP-ACT) were dissected on microscopic slides and fixed with 4% paraformaldehyde in phosphate-buffered saline (PBS). Longitudinal cross sections of midgut (MG) tissue, hindgut (HG) tissue and Malpighian tubules (MT) were prepared according to published procedures (35). The larvae were processed for tissue preparation after 24, 48 and 96 h of feeding. The cross sections were incubated with 0.165 μM Alex FluorR 488 phalloidin (Invitrogen) in PBS containing 1% BSA for 20 min. After incubation, the samples were washed with PBS three times. The fluorescence of stained actin was viewed in a confocal laser-scanning microscope (TCS SP5; Leica, http://www.leica.com). The excitation wavelength was 488 nm and the barrier filter BP 530 (band pass, 515-545 nm) was used.

### EXAMPLE 2: IN VIVO EVALUATION OF THE STRATEGY FOR DSRNA PRODUCTION IN THE PLASTIDS OF TOBACCO PLANTS—COMPARISON OF RNAI RESPONSES

To test the feasibility of stable dsRNA expression in plastids, we transformed the tobacco (Nicotiana tabacum) plastid genome with three different types of dsRNA constructs (FIG. 1A; FIG. 4C). In ptDP constructs, the dsRNA is generated by transcription from two convergent promoters. In ptSL constructs, the dsRNA is also produced from two convergent promoters, but each strand is additionally flanked by sequences forming stemloop-type secondary structures (that are known to increase RNA stability in plastids; 11). In ptHP constructs, hairpin-type dsRNA (hpRNA) is produced by transcription of two transgene copies arranged as an inverted repeat (FIG. 1A).

As insect target genes, the ACT and SHR genes from the Colorado potato beetle (Leptinotarsa decemlineata; CPB), a notorious insect pest of potato and other Solanaceous plants, were chosen based on their high efficacy in inducing mortality in feeding assays with in vitro-synthesized dsRNAs (12, 3). ACT encodes β-actin, an essential cytoskeletal protein, and SHR encodes Shrub (also known as Vps32 or Snf7), an essential subunit of a protein complex involved in membrane remodeling for vesicle transport. To also test longer dsRNAs and test for a possible synergistic action, we additionally produced an ACT+SHR fusion gene. To preliminarily compare the RNAi responses to these dsRNAs in CPBs, we synthesized dsRNAs (ACT, SHR, ACT+SHR fusion and GFP as a control) by in vitro transcription and painted them onto young potato leaves. Second instar CPB larvae were then allowed to feed on these leaves for up to 9 days. All three insect gene-derived dsRNAs strongly reduced the growth of CPB larvae (FIG. 5). The ACT dsRNA was slightly more effective than the SHR dsRNA, whereas the ACT+SHR dsRNA was significantly less effective than either the ACT or SHR dsRNAs (FIG. 5), indicating that targeting two insect genes with the same dsRNA does not necessarily enhance insecticidal activity.

The initial in vivo evaluation of the three strategies for dsRNA production (ptDP, ptSL and ptHP constructs; FIGS. 1A and 4C) was performed with the ACT+SHR fusion gene in tobacco plants, because chloroplast transformation is relatively routine in this species. Transplastomic tobacco lines were produced by particle gun-mediated chloroplast transformation and purified to homoplasmy by additional rounds of regeneration and selection. Stable integration of the transgenes into the plastid genome via homologous recombination and successful elimination of all wild-type copies of the highly polyploid plastid genome were confirmed by RFLP analyses and inheritance assays (FIG. 1B; FIG. 6A). All transplastomic lines (referred to as Nt-ptDP-ACT, Nt-ptSL-ACT and Nt-ptHP-ACT lines) displayed no visible phenotype and were indistinguishable from wild-type plants, both under in vitro culture conditions and upon growth in the greenhouse (FIG. 6D), indicating that dsRNA expression in the chloroplast is phenotypically neutral.

To test if dsRNAs stably accumulate in chloroplasts, northern blot analyses were performed. The results revealed that all three types of expression constructs triggered production of substantial amounts of long dsRNAs (FIG. 1C), suggesting absence of efficient dsRNA-degrading mechanisms from plastids. dsRNA accumulation levels in Nt-ptDP plants and Nt-ptSL plants were very similar, indicating that the terminal stemloop-structures added to the ptSL constructs do not appreciably increase dsRNA stability (FIG. 1A, 1C). dsRNA accumulation levels in Nt-ptHP lines were even higher, but included significant amounts of shorter-than-expected transcripts (asterisk in FIG. 1C), possibly due to difficulties of the plastid RNA polymerase with transcribing sequences containing large inverted repeats. Therefore, we used the simple convergent promoter approach (ptDP constructs) for dsRNA expression in all subsequent experiments.

### EXAMPLE 3: STABLE PLASTID TRANSFORMATION IN POTATO—LONG DSRNAS ACCUMULATE TO HIGH LEVELS IN LEAVES OF TRANSPLASTOMIC POTATO LINES

We next optimized a protocol for biolistic plastid transformation in potato (13; see Example 1: Materials and Methods), the main host plant of CPB. The three target gene constructs (ACT, SHR and ACT+SHR, integrated into the ptDP cassette; FIG. 1A) were then introduced into the potato plastid genome by stable transformation. Homoplasmic transplastomic lines were isolated and three lines per construct (St-ptDP-ACT, St-ptDP-SHR and St-ptDP-ACT+SHR lines) were chosen for further analysis (FIG. 4B, 4E). To be able to compare the level of protection from herbivory in transplastomic and transgenic plants, the identical transgenes were introduced (as classical hairpin constructs) into the nuclear genome by Agrobacterium-mediated transformation (FIG. 4D; St-nuHP lines). Phenotypic analyses showed that all transplastomic and transgenic potato plants were indistinguishable from wild-type plants with regard to growth (under heterotrophic and autotrophic conditions) and tuber production (FIGS. 6C, 6E and 7).

Northern blot analyses of transplastomic potato lines revealed that the accumulation levels of ACT dsRNAs were higher than those of SHR and ACT+SHR dsRNAs (FIG. 1D). To determine the dsRNA amounts in leaves of the transplastomic plants, a dilution series of in vitro synthesized RNA was compared to extracted plant total RNA. This analysis revealed dsRNA accumulation levels in leaves of approximately 0.4% of the total cellular RNA for ACT, 0.05% for SHR and 0.1% for ACT+SHR (FIG. 1E). By contrast, hybridization signals were hardly detectable in the nuclear transgenic plants, consistent with efficient degradation of dsRNAs into small siRNAs by the plant's endogenous RNAi machinery, even in the best-expressing transgenic lines (FIGS. 8 and 9).

Since CPB larvae and beetles feed on leaves but not on below ground potato tubers, only the leaves need to be protected from herbivory. The expression of most plastid genes is drastically down-regulated in non-photosynthetic tissues (14, 15), which made it possible to prevent dsRNA production in the tuber where the accumulation of transgene-derived RNA is unnecessary and perhaps also undesired by the consumer. Comparative analyses of dsRNA accumulation in leaves and tubers revealed that indeed, dsRNA levels in tubers are nearly undetectably low (FIG. 1F).

### EXAMPLE 4: DSRNA PRODUCTION IN THE CHLOROPLASTS OF POTATO PLANTS OFFERS PROTECTION AGAINST CPB

Having established that long dsRNAs accumulate to high levels in leaves of transplastomic potato lines, we next tested whether dsRNA production in the chloroplast offers protection against CPB. To this end, the mortality of first instar CPB larvae was determined upon feeding on detached leaves from wild-type, transplastomic and transgenic potato plants for 9 consecutive days (FIG. 2A). In addition, the weight of all surviving larvae was measured to follow their growth (FIG. 2B). The bioassays revealed that all transplastomic potato plants induced high mortality in CPB larvae (FIG. 2A). The most potent insecticidal activity was conferred by the ACT dsRNA-expressing transplastomic plants that caused 100% mortality within five days. This is consistent with the high expression level of ACT dsRNA (FIG. 1E) and the high efficacy of in vitro synthesized ACT dsRNA (FIG. 5). By contrast, none of the nuclear transgenic potato plants conferred significant larval mortality (FIG. 2A), in line with the earlier finding that short siRNAs fed to insects have only small effects or do not induce an RNAi response at all (3). However, all nuclear transgenic lines caused reduced growth of CPB larvae (FIG. 2B), presumably due to the small amounts of dsRNAs the plants accumulate and the low efficiency of siRNAs in inducing gene silencing in the insect (FIGS. 8 and 9). While none of the CPB larvae survived feeding on transplastomic St-ptDP-ACT plants, some of the larvae survived for 9 days on St-ptDP-SHR and St-ptDP-ACT+SHR leaves. However, these survivors displayed a very strong growth retardation (FIG. 2B).

To confirm that the killing of the CPB larvae by the transplastomic plants was due to induction of RNAi, expression of the target genes was determined in the gut of CPB larvae after three days of feeding (i.e., when the larvae fed on the transplastomic plants were still alive). Already at this early stage, expression of β-actin and Shrub was strongly suppressed in the insects (FIG. 2C, 2D). As expected based on the mortality data (FIG. 2A), target gene suppression was strongest in larvae fed on St-ptDP-ACT plants (FIG. 2C).

CPB resistance of transplastomic potato plants was further assessed by determining the leaf area consumed by CPB larvae and adult beetles. Almost no visible consumption of leaf biomass occurred in St-ptDP-ACT leaves (FIG. 3A), consistent with the rapid death of all larvae feeding on theses leaves (FIG. 2A). Similarly, adult beetles caused very little damage to transplastomic St-ptDP-ACT leaves (FIG. 3B). Finally, whole plants were also exposed to second instar larvae (which are generally less sensitive to insecticidal agents than first instar larvae) and survival was scored (FIG. 3C). This test resulted in ˜10% survival of the larvae after 6 days of feeding on St-ptDP-ACT plants (and ˜60% survival upon feeding on St-ptDP-SHR plants), presumably due to the initial larval growth and development on wild-type leaves. However, the larvae grew very poorly after transfer to the transplastomic plants and the damage they caused to the leaves was very small (FIG. 11). It is important to note that, in nature, CPB larvae typically hatch and feed on the same plant and, therefore, would not enjoy a wild-type diet prior to feeding on the transplastomic plant.

Our data reported here underscore the importance of producing large amounts of long dsRNAs to achieve efficient plant protection. While transplastomic ACT dsRNA-expressing plants cause 100% lethality to CPB larvae, SHR dsRNA-expressing plants are somewhat less efficient (70% mortality after 9 days; FIG. 2A). This correlates with significantly lower accumulation levels of the SHR dsRNA. Since both constructs are driven by the same expression signals, we conclude that the SHR sequence chosen is less stable in plastids than the ACT sequence. Consequently, testing other fragments of the SHR gene seems an appropriate future strategy to further improve the insecticidal efficiency of SHR dsRNA-expressing transplastomic plants.

### EXAMPLE 5: PLASTID-EXPRESSED ACT DSRNA SILENCES THE ACTIN GENE IN CPB

To ultimately confirm that the plastid-expressed ACT dsRNA silences the actin gene in CPB larvae, we examined actin filaments in the larval midgut, hindgut and Malpighian tubules by staining with FITC-labeled phalloidin. Already after 1-2 days of feeding on transplastomic leaves, the larvae displayed disorganized actin filaments, which were particularly obvious in the columnar cells of the midgut (FIG. 12). Also, the intensity of phalloidin-FITC labeling progressively decreased with the time of feeding (FIG. 12), strongly suggesting that actin deficiency is the cause of death in the larvae.

Moreover, accumulation of ACT-derived siRNAs was detected in gut tissue of larvae fed with transplastomic leaves, whereas accumulation in larvae fed with nuclear transgenic leaves was below the limit of reliable detection (FIG. 2E).

The present invention refers to the following nucleotide sequences:

SEQ ID No. 1:
Nucleotide sequence of the expression cassette of the construct Nt-ptDP-
ACT + SHR, the sequences which encode the dsRNA representing the CPB ACT
and SHR genes are underlined:
gctcccccgccgtcgttcaatgagaatggataagaggctcgtgggattgacgtgagggggcagggatggctatattt
ctgggagcgaactccgggcgaatacgaagcgcttggatacgcatgcctgcaggcacgaggtttttctgtctagtgag
cagtgtccaacctcaaaagacaacatgtgtgacgacgatgtagcggctcttgtcgtagacaatggatccggtatgtg
caaagccggtttcgcaggagatgacgcaccccgtgccgtcttcccctcgatcgtcggtcgcccaaggcatcaagg
agtcatggtcggtatgggacaaaaggactcatacgtaggagatgaagcccaaagcaaaagaggtatcctcaccc
tgaaataccccatcgaacacggtatcatcaccaactgggatgacatgcaatgtcatccatcatgtcgtgtacattgtc
cacgtccaagtttttatcgctttcttaagagcttcagctgcatttttcatagattccaatactgtggtgttcgtactagctcc
ctccagagcttctcgttgaagttcaatagtagttaaagtgccatctatttgcaactgatttttttctaatcgcttcttccgcttc
agcgcttgcatggccgctcagatccccgggtaccgagctcgtatccaagcgcttcgtattcgcccggagttcgctccc
agaaatatagccatccctgccccctcacgtcaatcccacgagcctcttatccattctcattgaacgacggcggggga
gc
SEQ ID No. 2:
Nucleotide sequence of the expression cassette of the construct Nt-ptSL-ACT + SHR,
the sequences which encode the dsRNA representing the CPB ACT
and SHR genes are underlined:
gctcccccgccgtcgttcaatgagaatggataagaggctcgtgggattgacgtgagggggcagggatggctatattt
ctgggagcgaactccgggcgaatacgaagcgcttggatacgcatgcgggtgggtggaaaaccacccacccctg
caggcacgaggtttttctgtctagtgagcagtgtccaacctcaaaagacaacatgtgtgacgacgatgtagcggctct
tgtcgtagacaatggatccggtatgtgcaaagccggtttcgcaggagatgacgcaccccgtgcggtcttcccctatat
cgtcggtcgcccaaggcatcaaggagtcatggtcctatcgacaaaaggactcatacgtaggagatgaagccc
aaagcaaaagaggtatcctcaccctgaaataccccatcgaacacctatcatcaccaactcgatgacatgcaat
gtcatccatcatgtcgtgtacattgtccacgtccaagtttttatgggctttcttaagagcttcagctgcatttttcatagattcc
aatactgtggtgttcgtactagctccctccagagcttctcgttgaagttcaatagtagttaaagtgccatctatttgcaact
gatttttttctaatcgcttcttccgcttcagcgcttgcatggcccctcagatcccgcacgctcctaatggagcgtgcggtat
ccaagcgcttcgtattcgcccggagttcgctcccagaaatatagccatccctgccccctcacgtcaatcccacgagc
ctcttatccattctcattgaacgacggcgggggagc
SEQ ID No. 3:
Nucleotide sequence of the expression cassette of the construct Nt-ptHP-ACT + SHR,
the sequences which encode the dsRNA representing the CPB ACT
and SHR genes are underlined:
gctcccccgccgtcgttcaatgagaatggataagaggctcgtgggattgacgtgagggggcagggatggctatattt
ctgggagcgaactccgggcgaatacgaagcgcttggatacgcatgcctgcaggcacgaggtttttctgtctagtgag
cagtgtccaacctcaaaagacaacatgtgtgacgacgatgtagcggctcttgtcgtagacaatggatccggtatgtg
caaagccggtttcgcaggagatgacgcaccccgtgccgtcttcccctcgatcgtcggtcgcccaaggcatcaagg
agtcatggtcctatcgacaaaaggactcatacgtaggagatgaagcccaaagcaaaagaggtatcctcaccc
tgaaataccccatcgaacacggtatcatcaccaactgggatgacatgcaatgtcatccatcatgtcgtgtacattgtc
caccttccaagtttttatgggctttcttaagagcttcagctgcatttttcatagattccaatactgtggtgttcgtactagctcc
ctccagagcttctcgttgaagttcaatagtagttaaagtgccatctatttgcaactgatttttttctaatcgcttcttccgcttc
agcgcttgcatggccctctcagatctggtacggaccgtactactctattcgtttcaatatatttatttgtttcagctgactgca
agattcaaaaatttctttattattttaaattttgtgtcactcaaaaccagataaacaatttgatatagaggcactatatatat
acatattctcgattatatatgtaaatgagttaaccttttttttccacttaaaattatatagggggatccccggggagcccc
atgcaagcgctgaagcggaagaagcgattagaaaaaaatcagttgcaaatagatggcactttaactactattgaa
cttcaacgagaagctctggagggagctagtacgaacaccacagtattggaatctatgaaaaatgcagctgaagct
cttaagaaagcccataaaaacttggacgtggacaatgtacacgacatgatggatgacattgcatgtcatcccagttg
gtgatgatactttgttcgatggggtatttcagggtgaggatacctcttttgctttgggcttcatctcctaCgtatgagtccttt
tgtcccataccgaccatgactccttgatgccttgggcgaccgacgatcgaggggaagacggcacggggtgcgtca
tctcctgcgaaaccggctttgcacataccggatccattgtctacgacaagagccgctacatcgtcgtcacacatgttgt
cttttgaggttggacactgctcactagacagaaaaacctcgtgcgagctcatcaaataaaacgaaaggctcagtcg
aaagactgggcctttcgttttatctgttgtttgtcggtgaacgctctcctgagtaggacaaatccgccgggagcggattt
gaacgttgcgaagcaacggcccggagggtggcgggcaggacgcccgccataaactgccaggcatcaaattaa
gcagaaggccatcctgacggatggcctttttgcgtttctac
SEQ ID No. 4:
Nucleotide sequence of the expression cassette of the construct St-ptDP-ACT, the
sequence which encodes the dsRNA representing the CPB ACT gene is
underlined:
gctcccccgccgtcgttcaatgagaatggataagaggctcgtgggattgacgtgagggggcagggatggctatattt
ctgggagcgaactccgggcgaatacgaagcgcttggatacgcatgcctgcaggcacgaggtttttctgtctagtgag
cagtgtccaacctcaaaagacaacatgtgtgacgacgatgtagcggctcttgtcgtagacaatggatccggtatgtg
caaagcccgtttcgcaggagatgacgcccccgtcttgcccctcttcccctcctatcgtcggtcgcccaaggcatcaagg
agtcatggtcctatcgacaaaaggactcatacgtaggagatgaagcccaaagcaaaagaggtatcctcaccc
tgaaataccccatcgaacacccgtatcatcaccaactgggatgacatgagctcgtatccaagcgcttcgtattcgccc
ggagttcgctcccagaaatatagccatccctgccccctcacgtcaatcccacgagcctcttatccattctcattgaacg
acggcgggggagc
SEQ ID No. 5:
Nucleotide sequence of the expression cassette of the construct St-ptDP-SHR, the
sequence which encodes the dsRNA representing the CPB SHR gene is
underlined:
gctcccccgccgtcgttcaatgagaatggataagaggctcgtgggattgacgtgagggggcagggatggctatattt
ctgggagcgaactccgggcgaatacgaagcgcttggatacgcatgcctgcaggcaatgtcatccatcatgtcgtgt
acattgtccacgtccaagtttttatgggctttcttaagagcttcagctgcatttttcatagattccaatactgtggtgttcgtac
tagctccctccagagcttctcgttgaagttcaatagtagttaaagtgccatctatttgcaactgatttttttctaatcgcttctt
cccgcttcaggtcttqcatctttccctctcgagctcgtatccaagcgcttcgtattcgcccggagttcgctcccagaaatat
agccatccctgccccctcacgtcaatcccacgagcctcttatccattctcattgaacgacggcgggggagc
SEQ ID No. 6:
Nucleotide sequence of the expression cassette of the construct St-ptDP-ACT + SHR,
the sequences which encode the dsRNA representing the CPB ACT
and SHR genes are underlined:
gctcccccgccgtcgttcaatgagaatggataagaggctcgtgggattgacgtgagggggcagggatggctatattt
ctgggagcgaactccgggcgaatacgaagcgcttggatacgcatgcctgcaggcacgaggtttttctgtctagtgag
cagtgtccaacctcaaaagacaacatgtgtgacgacgatgtagccctcttgtcgtagacaatggatccctatgtg
caaagccggtttcgcaggagatgacgcaccccttgctttcttcccctcgatcgtcggtcgcccaaggcatcaagg
agtcatggtcggtatgggacaaaaggactcatacgtaggagatgaagcccaaagcaaaagaggtatcctcaccc
tgaaataccccatcgaacacggtatcatcaccaactgggatgacatgcaatgtcatccatcatgtcgtgtacattgtc
cacccttccaagtttttatgggctttcttaagagcttcagctgcatttttcatagattccaatactgtggtgttcgtactagctcc
ctccagagcttctcgttgaagttcaatagtagttaaagtgccatctatttgcaactgatttttttctaatcgcttcttccgcttc
agcgcttgcatggccgctcagatccccgggtaccgagctcgtatccaagcgcttcgtattcgcccggagttcgctccc
agaaatatagccatccctgccccctcacgtcaatcccacgagcctcttatccattctcattgaacgacggcggggga
gc
SEQ ID No. 7: Phytophthora infestans (potato blight) sequences
Nucleotide sequence of the expression cassette of the construct St-ptDP-EPI + PMA,
the sequences which encode the dsRNA representing the Phytophthora
infestans EPI and PMA genes are underlined:
gctcccccgccgtcgttcaatgagaatggataagaggctcgtgggattgacgtgagggggcagggatggctatattt
ctgggagcgaactccgggcgaatacgaagcgcttggatacgcatgcctgcagaaagaaggaagtcacgccaga
ggacacggagctgctccagaaggcgcagagcaatgtgagcgcatacaacagcgacgtcacctcgcgcatctgct
acctgaaggtcgacagtctcgagactcaagtcgtctccgcgagaactacaagttccacgtttcccttgcagcgtc
aactccacaaggagctcgcggctgtgccaatcagaattgcgagtcatccaagtacgacatcgtcatctactcgc
agtcgtggaccaacacgctgaaggtgacgtcgattacgcccgccaacgctggtgccgcaggtaactcgtacatgtc
catggcgacgcccaacgacgtcaagaactacacgaacgacgttggccagatccagtgggcgcaggtgccgctg
aacgccgcgcttgacaagctcaagtcgtcccgtgagggtctgacatccgatgaggctgagaagcgtctggccgag
tacggcccgaacaagctgccggaggagaaggtgaacaagctgacgctgttcctgggcttcatgtggaacccgctg
tcgtgggccatggaggtggccgctttctgtcgattgtgctgctggattacctctgatttcgcgctgatcctgttcctgctg
ctgctaaacagatctcccgggtaccgagctcgtatccaagcgcttcgtattcgcccggagttcgctcccagaaatata
gccatccctgccccctcacgtcaatcccacgagcctcttatccattctcattgaacgacggcgggggagc

The Present Invention Refers to the Following Tables:

TABLE 1
List of oligonucleotides used in the context of the invention. Recognition sequences of introduced restriction sites are underlined.
The T7 promoter sequence is indicated in italics.

| | | SEQ | |
|---|---|---|---|
| | | ID | |
| Oligonucleotide | Sequence 5′-3′ | No. | Description and Use |
| Prrn(HindIII)-F | AAAAAAGCTTGCTCCCCCGCCGTCGTTC | 8 | forward primer for amplification of the Prm promoter; introducing a HindlII site |
| Prrn(SphI)-R | AAAAGCATGCGTATCCAAGCGCTTCGTAT | 9 | reverse primer for amplification of the Prm promoter; introducing an SphI site |
| Prrn(EcoRI)-F | AAAAGAATTCGCTCCCCCGCCGTCGTTC | 10 | forward primer for amplification of the Prm promoter; introducing an EcoRI site |
| Prrn(SacI)-R | AAAAGAGCTCGTATCCAAGCGCTTCGTAT | 11 | reverse primer for amplification of the Prm promoter; introducing a SacI site |
| PrrnSL1(PstI)-R | AAAACTGCAGGGGTGGGTGGTTTTCCACCC | 12 | forward primer for amplification of the Prm promoter; introducing a PstI site and stem- |
| | ACCCGTATCCAAGCGCTTCGTAT | | loop sequence 1 |
| PrrnSL2(BamHI)-R | AAAAGGATCCCGCACGCTCCATTAGGAGCGT | 13 | reverse primer for amplification of the Prm promoter; introducing a BamHI site and |
| | GCGGTATCCAAGCGCTTCGTAT | | stem-loop sequence 2 |
| actin(SbfI)-F | AAACCTGCAGGCACGAGGTTTTTCTGTCTAG | 14 | forward primer for amplification of the ACT gene fragment; introducing an Sbfl site |
| actin(SacI)-R | AAAAGAGCTCATGTCATCCCAGTTGGTGAT | 15 | reverse primer for amplification of the ACT gene fragment; introducing a Sad 1 site |
| shrub(SbfI)-F | AAAACCTGCAGGCAATGTCATCCATCATGTC | 16 | forward primer for amplification of the SHR gene fragment; introducing an Sbfl site |
| | G | | |
| shrub(SacI)-R | AAAAGAGCTCGAGCGGCCATGCAAGC | 17 | reverse primer for amplification of the SHR gene fragment; introducing a Sad 1 site |
| TrrnB(SacI)-F | AAAAGAG19CTCATCAAATAAAACGAAAGGCT | 18 | forward primer for amplification of the E. coli rmB terminator; introducing a SacI site |
| | CAGTCG | | |
| TrrnB(EcoRI)-R | AAAAGAATTCGTAGAAACGCAAAAAGGCCAT | 19 | reserve primer for amplification of the E. coli rmB terminator; introducing an EcoRI |
| | CC | | site |
| act + shr(SacI)-F | AAAAGAGCTCGCACGAGGTTTTTCTGTC | 20 | forward primer for amplification of the ACT+30SHR fragment; introducing a SacI site |
| act + shr(SmaI)-R | AAAACCCGGGAGCGGCCATGCAAGC | 21 | reverse primer for amplification of the ACT+30SHR fragment; introducing a SmaI site |
| actin(XbaI)-F | AAAATCTAGACACGAGGTTTTTCTGTCTAG | 22 | forward primer for amplification of the ACT gene fragment; introducing an XbaI site |
| actin(XhoI)-F | AAAACTCGAGCACGAGGTTTTTCTGTCTAG | 23 | forward primer for amplification of the ACT gene fragment; introducing an XhoI site |
| actin(BgIII)-R | AAAAGATCTATGTCATCCCAGTTGGTGAT | 24 | reverse primer for amplification of the ACT gene fragment; introducing a BglI site |
| shrub(XbaI)-F | AAAATCTAGACAATGTCATCCATCATGTCG | 25 | forward primer for amplification of the SHR gene fragment; introducing an XbaI site |
| shrub(XhoI)-F | AAAACTCGAGCAATGTCATCCATCATGTCG | 26 | forward primer for amplification of the SHR gene fragment; introducing an XhoI site |
| shrub(BamHI)-R | AAAAGGATCCGAGCGGCCATGCAAGC | 27 | reverse primer for amplification of the SHR gene fragment; introducing a BamHI site |
| PBT7actshrFwd | TAATACGACTCACTATAGGCCTGCAGGCACG | 28 | forward primer for amplification of the ACT+30SHR fragment introducing the T7 |
| | AGGTTTTTCTGT | | promoter sequence; for in vitro dsRNA synthesis |
| PBT7actshrRev | TAATACGACTCACTATAGGGGCCCGGGATCC | 29 | reverse primer for amplification of the ACT+30SHR fragment introducing the T7 |
| | GATATGCC | | promoter sequence; for in vitro dsRNA synthesis |
| PBT7actFwd | TAATACGACTCACTATAGGATGTGTGACGAC | 30 | forward primer for amplification of the ACT fragment; introducing the T7 promoter |
| | GATGTAGCG | | sequence; for in vitro dsRNA synthesis |
| PBT7actRev | TAATACGACTCACTATAGGTTCCATGTCATCC | 31 | reverse primer for amplification of the ACT fragment; introducing the T7 promoter |
| | CAGTTGG | | sequence; for in vitro dsRNA synthesis |
| PBT7 shrubFwd | TAATACGACTCACTATAGGGAGTGGCCCTGC | 32 | forward primer for amplification of the SHR fragment; introducing the T7 promoter |
| | AAGCCCTCAA | | sequence; for in vitro dsRNA synthesis |
| PBT7shrubRev | TAATACGACTCACTATAGGGCAATGTCATCC | 33 | reverse primer for amplification of the SHR fragment; introducing the T7 promoter |
| | ATCATGTC | | sequence; for in vitro dsRNA synthesis |
| T7GFPfwd | TAATACGACTCACTATAGGAGGACGACGGCA | 34 | forward primer for amplification of the gfp gene; introducing the T7 promoter |
| | ACTACAAG | | sequence; for in vitro dsRNA synthesis |
| T7GFPrev | TAATACGACTCACTATAGGCTGGGTGCTCAG | 35 | reverse primer for amplification of the gfp gene; introducing the T7 promoter |
| | GTAGTGGT | | sequence; for in vitro dsRNA synthesis |
| PBactinReTiFwd | CCAGTCCTCCTCACTGAAGC | 36 | forward primer for gRT-PCR analysis of ACT expression |
| PBactinReTiRev | ACGACCAGAAGCGTACAAGG | 37 | reverse primer for gRT-PCR analysis of ACT expression |
| PBshrubReTiFwd | GATGATTTGGACGATGCTGA | 38 | forward primer for gRT-PCR analysis of SHR expression |
| PBshrubReTiRev | TAGCTGGTTTGACTGGCTTG | 39 | reverse primer for gRT-PCR analysis of SHR expression |
| PBReTiRps18Fwd | GCGGGAGAATGTACAGAGGA | 40 | forward primer for gRT-PCR analysis of RPS18 expression (as reference gene) |
| PBReTiRps18Rev | AAGTCTTCACGGAGCTTGGA | 41 | reverse primer for gRT-PCR analysis of RPS18 expression (as reference gene) |
| PBRP4ReTiFwd | CGTCAAAGAAACGAGCATTG | 42 | forward primer for gRT-PCR analysis of RPS4 expression (as reference gene) |
| PBRP4ReTiRev | TCGCTGACACTGTAGGGTTG | 43 | reverse primer for gRT-PCR analysis of RPS4 expression (as reference gene) |

The present invention refers to the following (additional) references:
1. Baum, Nat. Biotechnol. 25, 1322 (2007)
2. Mao, Nat. Biotechnol. 25, 1307 (2007)
3. Bolognesi, PLoS One 7, e47534 (2012)
4. Whyard, Insect Biochem. Mol. Biol. 39, 824 (2009)
5. Zha, PLoS One 6, e20504 (2011)
6. Pitino, PLoS One 6, e25709 (2011)
7. Kumar, PLoS One 7, e31347 (2012)
8. Zhu, PLoS One 7, e38572 (2012)
9. Voinnet, Cell 136, 669 (2009)
10. Kumar, J. Insect Physiol. 55, 273 (2009)
11. Stern, Cell 51, 1145 (1987)
12. Zhu, Pest. Manag. Sci. 67, 175 (2010)
13. Valkov, Transgenic Res. 20, 137 (2011)
14. Kahlau, Plant Cell 20, 856 (2008)
15. Valkov, Plant Physiol. 150, 2030 (2009)
16. Price, Trends Biotechnol. 26, 393 (2008)
17. Gahan, Science 293, 857 (2001)
18. Griffitts, Science 293, 860 (2001)
19. Gassmann, Proc. Natil. Acad. Sci. USA 111, 5141 (2014)
20. Zhou, Plant J. 52, 961 (2007)
21. Oey, Plant J. 57, 436 (2009)
22. Murashige, Physiol. Plant. 15, 473 (1962)
23. Zhou, Plant Biotechnol. J. 6, 897 (2008)
24. Zou, Mol. Gen. Genomics 269, 340 (2003)
25. Scharff, Plant J. 50, 782 (2007)
26. Chen, Plant J. 36, 731 (2003)
27. Birch-Machin, Plant Biotechnol. J. 2, 261 (2004)
28. Svab, Proc. Natl. Acad. Sci. USA 90, 913 (1993)
29. Rocha-Sosa, EMBO J. 8, 23 (1989)
30. Doyle, Focus 12, 13 (1990)
31. Wurbs, Plant J. 49, 276 (2007)
32. Zhou, Nucleic Acids Res. 41, 3362 (2013)
33. Grover Jr., Arch. Insect Biochem. Physiol. 8, 59 (1988)
34. Mittapalli, J. Insect Sci. 7, 1 (2007)
35. Kaltenpoth., Physiol. Entomol. 35, 196 (2010)

1. A plant comprising a plastid comprising a double-stranded RNA (dsRNA) capable of silencing at least one target gene of a pest of a plant (plant pest) or of an agent causing a disease of a plant (plant pathogen),
wherein said dsRNA comprises two complementary single-stranded RNA strands.

2. The plant of claim 1, wherein said dsRNA comprises two separate complementary single-stranded RNA strands.

3. The plant of claim 1, wherein said plastid is a chloroplast.

4. The plant of claim 1 which is a vascular plant.

5. The plant of claim 1, wherein the sense strand of said dsRNA is at least 60% identical to an RNA transcribed from a nucleotide sequence of at least 50 contiguous nucleotides of said target gene.

6. The plant of claim 1, wherein said plastid is genetically engineered so as to comprise a nucleotide sequence encoding said dsRNA, wherein said dsRNA is transcribed from said nucleotide sequence.

7. The plant of claim 1, wherein said dsRNA is expressed by transcription from a nucleotide sequence flanked by two convergent promoters.

8. The plant of claim 1, wherein said plant pest or plant pathogen is selected from the group consisting of:
(i) an insect;
(ii) a nematode;
(iii) a mollusk; and
(iv) a fungal plant pathogen.

9. The plant of claim 8, wherein said insect is a Colorado potato beetle (Leptinotarsa decemlineata), including any juvenile stage of said beetle.

10. The plant of claim 8, wherein said fungal plant pathogen is Phytophthora infestans.

11. The plant of claim 1, which is a potato plant or a tobacco plant.

12. The plant of claim 1, wherein said target gene is ACT, SHR, EPIC2B or PnPMA1.

13. A plastid as defined in claim 1.

14. A plant cell comprising a plastid of claim 13.

15. A method of controlling a plant pest or a plant pathogen as defined in claim 1 and/or of protecting a plant from said plant pest or plant pathogen comprising the steps of
(i) growing a plant of claim 1; and
(ii) allowing said plant pest or plant pathogen to affect said plant.
